(19) United States
(12) Patent Application Publication
(10) Pub. No.: US 2024/0082313 A1
(43) Pub. Date: Mar. 14, 2024

(54) PHARMACEUTICAL COMPOSITIONS AND THEIR METHODS OF USE

(71) Applicant: The Trustees of Indiana University, Bloomington, IN (US)

(72) Inventor: Hiroki Yokota, Bloomington, IN (US)

(21) Appl. No.: 18/262,799

(22) PCT Filed: Jan. 25, 2022
(86) PCT No.: PCT/US2022/013663

§ 371 Date: Jul. 25, 2023

Related U.S. Application Data
(60) Provisional application No. 63/141,665, filed on Jan. 26, 2021.

(51) Int. Cl. A61K 35/32; A61P 35/00; C12N 5/09

(52) U.S. Cl. CPC A61K 35/32; A61P 35/00; C12N 5/0693; C12N 2500/90; C12N 2501/415; C12N 2501/603; C12N 2501/606

(57) ABSTRACT

A pharmaceutical composition includes a cell-free conditioned medium (CM) or extract or concentrate thereof obtained from a mammalian cell culture medium comprising a cultured substantially homogenous cancerous mammalian cell population. At least a portion of the cancerous mammalian cell population is contacted by at least one small molecule cell growth signaling pathway activator before being cultured in the cell culture medium.

### CROSS-REFERENCE TO RELATED APPLICATIONS

This application claims priority to U.S. Provisional Patent Application No. 63/141,665, which was filed Jan. 26, 2021, the entire content of which is incorporated by reference herein.

### STATEMENT ON FEDERALLY SPONSORED RESEARCH OR DEVELOPMENT

This invention was made with government support under AR052144 and CA238555 awarded by National Institutes of Health. The Government has certain rights in the invention.

### FIELD

Embodiments disclosed herein are directed to conditioned media compositions and to their use to treat cancers.

### BACKGROUND

Cancer is characterized by proliferation of abnormal cells. Many cancer treatments include painful surgeries and chemotherapies with undesirable side effects. An ongoing, urgent need exists for new therapeutic interventions for cancer. The present subject matter addresses this need.

### SUMMARY

In one aspect, the disclosure relates to pharmaceutical composition of a cell-free conditioned medium (CM) or extract or concentrate thereof obtained from a mammalian cell culture medium containing a cultured substantially homogenous cancerous mammalian cell population where a portion of the cancerous mammalian cell population is contacted by a small molecule cell growth signaling pathway activator before being cultured in the cell culture medium.

In another aspect, the disclosure relates to a kit comprising: a) a pharmaceutical composition according to the preceding aspect; b) a container; c) a label; and d) instructions that provide methods for administering the composition to a subject in need thereof.

In another aspect, the disclosure relates to a method to treat a cancer in a subject in need thereof by administering to the subject in need thereof a therapeutically effective amount of a cell-free conditioned medium (CM) or extract or concentrate thereof obtained from a mammalian cell culture medium containing a cultured substantially homogenous cancerous mammalian cell population where a portion of the cancerous mammalian cell population is contacted by a small molecule cell growth signaling pathway activator before being cultured in the cell culture medium.

In another aspect, the disclosure relates to a method to decrease expression of a tumor-promoting gene in a cell by contracting the cell with a therapeutically effective amount of a cell-free conditioned medium (CM) or extract or concentrate thereof obtained from a mammalian cell culture medium containing a cultured substantially homogenous cancerous mammalian cell population where a portion of the cancerous mammalian cell population is contacted by a small molecule cell growth signaling pathway activator before being cultured in the cell culture medium.

In another aspect, the disclosure relates to a method to increase expression of a tumor-suppressing gene in a cell by contacting the cell with a therapeutically effective amount of a cell-free conditioned medium (CM) or extract or concentrate thereof obtained from a mammalian cell culture medium containing a cultured substantially homogenous cancerous mammalian cell population where a portion of the cancerous mammalian cell population is contacted by a small molecule cell growth signaling pathway activator before being cultured in the cell culture medium.

In another aspect, the disclosure relates to a process to produce a conditioned medium (CM) by contacting cancerous mammalian cells by a small molecule cell growth signaling pathway activator to generate pre-treated cancerous mammalian cells; culturing the pre-treated cancerous mammalian cells in a mammalian cell culture medium for a period of time sufficient to condition the medium; removing the pre-treated cancerous mammalian cells from the culture medium; and, collecting the conditioned medium.

In another aspect, the disclosure relates to a method to identify an anti-tumor property in a conditioned medium (CM) by contacting cancerous mammalian cells by a small molecule cell growth signaling pathway activator to generate pre-treated cancerous mammalian cells; culturing the pre-treated cancerous mammalian cells in a mammalian cell culture medium to condition the medium; removing the pre-treated cancerous mammalian cells from the culture medium; collecting the conditioned medium; and, assaying the collected conditioned medium for an anti-tumor property.

This summary is not intended to identify key or essential features of the claimed subject matter, nor is it intended to be used as an aid in limiting the scope of the claimed subject matter. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, references labels have been repeated among the figures to indicate corresponding or analogous elements.

FIG. 1 illustrates generation of iTS cells from osteocytes, osteoclasts, and mammary tumor cells. CM=conditioned medium, CN=control (no CM treatment), β-cat=β-catenin plasmids, A5=MLO-A5 osteocytes, RAW=RAW 264.7 osteoclasts, and EO=EO771 mammary tumor cells. The single, double, and triple asterisks indicate p<0.05, p<0.01, and p<0.0001, respectively. (A) Procedure for generating iTS cells by the overexpression of β-catenin. (B-D) Reduction in EdU-based proliferation and TRANSWELL® invasion of EO771 mammary tumor cells by A5 osteocyte-derived, RAW264.7 osteoclast-derived, and EO771 breast cancer-derived iTS CMs, respectively.

FIG. 2 illustrates generation of iTS cells from human and mouse cancer cell lines. CM=conditioned medium, CN=control (no CM treatment), β-cat=β-catenin plasmids, MDA=MDA-MB-231 breast cancer cells, PA=PANC-1 pancreas cancer cells, and PC=PC-3 prostate cancer cells. The double and triple asterisks indicate p<0.01, p<0.0001. (A) Effective anti-tumor linkage. (B-D) Inhibition of EdU-based proliferation, scratch-based migration, and TRANSWELL® invasion of MDA-MB-231 breast cancer cells, PANC-1 pancreatic cancer cells, and PC-3 prostate cancer cells by their own iTS-CMs, respectively.

FIG. 3 illustrates inhibition of the growth of cancer tissue fragments by tissue-derived iTS CM. CM=conditioned medium, pl=placebo, and β-cat=β-catenin plasmids. The double and triple asterisk indicates p<0.01 and 0.0001, respectively. (A) Procedure for the ex vivo tumor tissue assay. (B) Shrinkage of breast cancer tissue fragments by β-catenin-overexpressing and BML284-treated MCF7-derived iTS CM. (C) Shrinkage of prostate cancer tissue fragments by β-catenin-overexpressing and BML284-treated PC3-derived iTS CM. (D) Shrinkage of EO771 mammary tumor spheroid (green) by co-culturing with β-catenin-overexpressing EO771 spheroid (red) and by β-catenin-overexpressing E0771-derived iTS CM.

FIG. 4 illustrates inhibition of tumor invasion and tumor growth by iTS CM in the mouse model. CM=conditioned medium, pl=placebo, and β-cat=β-catenin plasmids. The single and triple asterisks indicate p<0.05 and p<0.0001, respectively. (A) Procedure for the extravasation assay for checking the invasion of tumor cells into the lung. (B) Increase in the invaded cells by inoculating β-catenin-overexpressing EO771 cells, and a decrease by the administration of β-catenin-overexpressing EO771 cell-derived CM. (C) Procedure for the inoculation of EO771 cells to the mammary fat pad. (D) Increase in tumor weight by inoculating β-catenin-overexpressing EO771 cells, and a decrease by the administration of β-catenin-overexpressing EO771 cell-derived CM. (E) Increase in tumor weight by the systemic administration of BML284, and a decrease by the administration of BME284-treated EO771 cell-derived CM.

FIG. 5 illustrates prevention of bone loss by b-catenin-overexpressing and BML284-treated EO771 cell-derived CM. pl=placebo, β-cat=β-catenin, BML=BML284, and CM=conditioned medium. The single and double asterisks indicate p<0.05 and 0.01, respectively. (A) Inoculation of EO771 mammary tumor cells into the tibia. (B) μCT images of the proximal tibia and BV/TV (bone volume ratio), BMD (bone mineral density), Tb.N (trabecular number), and Tb. Sp (trabecular separation) for 5 groups of C57BL/6 female mice. They are the placebo (no treatment), inoculation of β-catenin-overexpressing cells ((3-cat cells), administration of β-catenin-overexpressing EO771 cell-derived CM ((3-cat CM), inoculation of BML284-treated cells (BML), and administration of BML284-treated cell-derived CM (BML CM).

FIG. 6 illustrates mass spectrometry-based prediction of tumor suppressors and the effect of enolase 1 and ubiquitin C. CM=conditioned medium, CN=control (no CM treatment), and β-cat=β-catenin plasmids. The single and double asterisks indicate p<0.05 and p<0.01, respectively. (A) List of 25 top tumor suppressor candidates identified by mass spectrometry-based proteomics analysis. (B) Reduction in MTT-based proliferation of EO771 mammary tumor cells by 9 recombinant proteins. (C) Expression of enolase 1 and ubiquitin C in β-catenin-overexpressing and BML284-treated EO771 CMs. (D) Inhibition in the scratch-based migration of EO771 mammary tumor cells by enolase 1 and ubiquitin C. (E-G) Repressive effects of AP-III-a4, an inhibitor of enolase 1, on the proliferation and migration of EO771 cells by β-catenin overexpressing iTS CM.

FIG. 7 illustrates effect of silencing enolase 1 and ubiquitin C. CM=conditioned medium, CN=control (no CM treatment), β-cat=β-catenin plasmids, siEnol=Enolase 1 siRNA, siUbc=ubiquitin C siRNA, EO=EO771 mammary tumor cells, TR=TRAMP prostate cancer cells, and PA=PANC-1 pancreas cancer cells. The double asterisk indicates p<0.01. (A) siRNA-mediated knockdown of enolase 1, and ubiquitin C in EO771 breast cancer cells. (B&C) Promotion of MTT-based proliferation, and scratch-based migration of EO771 breast cancer cells by enolase 1 and ubiquitin C siRNA-treated CMs. (D-I) Effects of enolase 1 and ubiquitin C siRNAs. Silencing these two proteins significantly prevented the reduction in EdU-based proliferation and TRANSWELL® invasion of EO771, TRAMP, and PANC-1 cells by their own β-catenin-overexpressing iTS CMs.

FIG. 8 illustrates effects of enolase 1, ubiquitin C, and iTS CM on the expression of tumor-promoting and tumor-suppressing genes. CM=conditioned medium, CN=control (no CM treatment), β-cat=β-catenin plasmids, siEnol=Enolase 1 siRNA, siUbc=ubiquitin C siRNA, EO=EO771 mammary tumor cells. (A) Expression of MMP9, Runx2, Snail, p53, and TRAIL in response to enolase 1 and ubiquitin C in EO771 breast cancer cells. (B) Expression of MMP9, Runx2, Snail, p53, and TRAIL in response to iTS CM after silencing enolase 1 and ubiquitin C. (C) Expression of MMP9, Runx2, Snail, p53, and TRAIL in response to EO771 cell-derived CM that was treated with an inhibitor of enolase 1 (AP-III-a4). (D) Expression of MMP9, Runx2, Snail, p53, and TRAIL in response to β-catenin-overexpressing iTS CM impaired by siRNAs specific to enolase 1 and ubiquitin C. (E&F) Decreased expression of p53 and TRAIL in EO771 cells and E0771-derived CM by silencing of enolase 1 and ubiquitin C. (G) Expression of MMP9, Runx2, Snail, p53, TRAIL, and caspase 3 in EO771 mammary tumor cells in response to β-catenin-overexpressing and BML284 pre-treatment tumor cell-derived CM. (H) Low survival for cancer patients with a high transcript level of MMP9, Runx2, or Snail. (I) The proposed regulatory mechanism to inhibit tumor progression by iTS-CM.

FIG. 9 illustrates inhibition of migration and invasion by iTS cells. CM=conditioned medium, pCN=control plasmids, CN=control (no CM treatment), Neat=β-catenin plasmid, A5=MLO-A5 osteocytes, RAW=RAW 264.7 osteoclasts, and EO=EO771 mammary tumor cells. The double and triple asterisks indicate p<0.01, and p<0.0001, respectively. (A-C) Inhibition of scratch-based migration of EO771 mammary tumor cells by p-catenin-overexpressing RAW264.7 osteocyte-derived, A5 osteocyte-derived, and EO771 tumor cell-derived iTS CM, respectively. (D) Reduction in 3-dimensional spheroid growth of EO771 mammary tumor cells by EO771 cell-derived iTS CM.

FIG. 10 illustrates inhibition of proliferation, migration, and invasion by tumor cell-derived iTS CM. CM=conditioned medium, CN=control (no CM treatment), pβcat=β-catenin plasmids, and TR=TRAMP prostate tumor cells. The triple asterisk indicates p<0.0001. (A) Inhibition of EdU-based proliferation, scratch-based migration, and TRANSWELL® invasion of 4T1.2 mammary tumor cells by β-catenin-overexpressing 4T1.2 tumor cell-derived iTS CM. (B) Inhibition of EdU-based proliferation, scratch-based migration, and TRANSWELL® invasion of TRAMP prostate tumor cells by β-catenin-overexpressing TRAMP tumor cell-derived iTS CM.

FIG. 11 illustrates inhibition of proliferation and invasion by tumor cell-driven iTS CM. CM=conditioned medium, CN=control (no CM treatment), β—cat=β-catenin plasmids, EO=EO771 mammary tumor cells, 4T=4T1.2 mammary tumor cells, TR=TRAMP prostate tumor cells. MD=MDA-MB-231 breast cancer cells, PA=PANC-1 pancreas cancer cells, and PC=PC-3 prostate cancer cells, The triple asterisk indicates p<0.0001. Tumor cells to be inhibited include EO771 mammary tumor cells (A), 4T1.2 mammary tumor cells (B), TRAMP prostate cells (C), MDA-MB-231 breast cancer cells (D), PANC-1 pancreatic cancer cells (E), and PC-3 prostate cancer cells (F).

FIG. 12 illustrates inhibition of ex vivo tumor growth by iTS CM with negative controls. CM=conditioned medium, pβcat=β-catenin plasmids, and MDA=MDA-MB-231 breast cancer cells. The double and triple asterisk indicates p<0.01 and p<0.0001, respectively. (A) Shrinkage of breast cancer tissue fragments (estrogen receptor-negative) by β-catenin-overexpressing, and BML284-treated MDA-derived iTS CM. (B-D) Negative controls for BML284-treated tumor-derived iTS CM. No shrinkage of cancer fragments by direct treatment of three kinds of human cancer cells (estrogen receptor-positive and negative breast cancer tissues, and prostate cancer tissue, respectively) with BML284.

FIG. 13 illustrates inhibition of tumor invasion by iTS CM in the mouse model with EO771 mammary tumor cells. CM=conditioned medium, and p βcat=β-catenin plasmids. The single, double and triple asterisks indicate p<0.05, p<0.01, and p<0.0001, respectively. (A) Increase in the invaded cells by the systemic administration of BML284, and a decrease by the administration of BML-treated EO771 cell-derived CM. (B) Bodyweight on days 0 and 18 for mice with mammary tumors.

FIG. 14 illustrates effect of enolase 1 and ubiquitin C on TRAMP prostate tumor cells and PANC-1 pancreatic tumor cells. CN=control, Eno1=enolase 1, Ubc=ubiquitin C, CM=conditioned medium, p βcat=β-catenin plasmids, and Poma=Pomalidomide. The single, and double asterisks indicate p<0.05, and p<0.01, respectively. (A&B) Reduction in EdU-based proliferation and TRANSWELL® invasion of TRAMP prostate tumor cells and PANC-1 pancreatic tumor cells in response to enolase 1 and ubiquitin C. (C-F) Repressive effects of pomalidomide, an inhibitor of E3 ubiquitin ligase, on the reduced proliferation and invasion of EO771 cells by β-catenin overexpressing iTS CM.

FIG. 15 illustrates effects of p53 and TRAIL on Regulatory mechanisms involved in iTS CM. CM=conditioned medium, CN=control (no CM treatment), si=siRNA, β-cat=β-catenin plasmids, and EO=EO771 mammary tumor cells. (A) Expression of p53, and TRAIL in β-catenin-overexpressing tumor cell-derived CM. (B) Expression of MMP9, Runx2, and Snail of EO771 mammary tumor cells in response to p53-overexpressing tumor cells-derived CM. (C) Upregulation of MMP9, Runx2, and Snail of EO771 mammary tumor cells in response to p53 silencing tumor cell-derived CM. (D) Expression of cleaved-caspase 3 in response to TRAIL in EO771 tumor cells. (E) Decreased expression of cleaved caspase 3 in EO771 cells by TRAIL silencing tumor cell-derived CM.

FIG. 16 illustrates expression of tumor-promoting genes in response to β-catenin in the EO771 mammary tumor cells. CN=control (no CM treatment), β—cat=β-catenin plasmids, and EO=EO771 mammary tumor cells.

FIG. 17 illustrates tumor suppression in vitro by Oct4-overexpressing CM derived from 4T1.2 mammary tumor cells. The double asterisk indicates p<0.01. CN=control, CM=conditioned medium, Oct4=Oct4 plasmids, and siOct4=Oct4 siRNA. (A) Generation of Oct4-overexpressing 4T1.2 cell-derived CM with the ultracentrifugation for the isolation of exosomes. (B-D) Enhancement of the reduction in MTT-based viability by Oct4-overexpressing CM that excluded exosomes, and the reduction in EdU-based proliferation, scratch-based migration, and TRANSWELL® invasion of 4T1.2 cells by Oct4-overexpressing 4T1.2 cell-derived CM. (E-G) Elevation in EdU-based proliferation, scratch-based migration, and TRANSWELL® invasion of 4T1.2 cells by Oct4-silenced 4T1.2 cell-derived CM. (H) Time-dependent shrinkage of 4T1.2 tumor spheroids by Oct4-overexpressing tumor spheroids and their CM.

FIG. 18 illustrates tumor suppression in vitro by OAC2-treated CM, derived from 4T1.2 mammary tumor cells. The double asterisk indicates p<0.01. CN=control, and CM=conditioned medium, (A) Generation of OAC2-treated 4T1.2-derived CM. (B) Elevation of Oct4 in 4T1.2 cells by OAC2 treatment. (C&D) Reduction in MTT-based viability and tumor spheroids by OAC2-treated 4T1.2-derived CM. (E&F) Reduction in ex vivo breast cancer tissue fragments by Oct4-overexpressing and OAC2-treated tumor cell-derived CM.

FIG. 19 illustrates tumor suppression and bone protection in vivo by Oct4-overexpressing and OAC2-treated tumor cell-derived CMs. The double asterisk indicates p<0.01. pl=placebo. (A) Inoculation of 4T1.2 tumor cells to the mammary fat pad and the tibia of BALB/c mice, followed by the daily intravenous administration of CMs. (B) Significant reduction of mammary tumors by Oct4-overexpressing and OAC2-treated tumor cell-derived CMs. (C) Prevention of bone loss in the proximal tibia by Oct4-overexpressing and OAC2-treated tumor cell-derived CMs. BV/TV=bone volume ratio, BMD=bone mineral density, Tb.N=trabecular number, and Tb. Sp=trabecular separation. (D) Reduction in the tumor-invaded area by Oct4-overexpressing and OAC2-treated tumor cell-derived CMs.

FIG. 20 illustrates tumor suppression by c-Myc- and Oct4-overexpressing tumor cell-derived CMs. The double asterisk indicates p<0.01. CN=control, c-Myc=c-Myc plasmids, C+O=c-Myc and Oct4, CM=conditioned medium, and pl=placebo. (A-C) Elevation of c-Myc and Oct4 in 4T1.2 cells, and the reduction in MTT-based viability, scratch-based migration, and TRANSWELL® invasion by c-Myc- and Oct4-overexpressing tumor cell-derived CMs. (D) Significant reduction of mammary tumors in BALB/c mice by c-Myc- and Oct4-overexpressing tumor cell-derived CMs. (E) Prevention of bone loss in the proximal tibia of BALB/c mice by c-Myc- and Oct4-overexpressing tumor cell-derived CMs. BV/TV=bone volume ratio, BMD=bone mineral density, Tb.N=trabecular number, and Tb. Sp=trabecular separation.

FIG. 21 illustrates Yamanaka factors (Oct4, c-Myc, Sox2, and Klf4) and their effect on Kdm3a and the selected tumor-promoting genes (Lrp5, MMP9, Runx2, TGFβ, and Snail). CM=conditioned medium, Oct4=Oct4 plasmids, siOct4=Oct4 siRNA, c-Myc=c-Myc plasmids, and C+O=c-Myc and Oct4. (A) Plasmid-based overexpression and siRNA-based silencing of Yamanaka factors to generate tumor cell-derived CM. (B) Downregulation of Kdm3a and the tumor-promoting genes by Oct4 overexpression and their upregulation by Oct4 silencing in 4T1.2 cells. (C&D) Downregulation of Kdm3a and the tumor-promoting genes by c-Myc and Oct4 overexpression, as well as OAC2 treatment in 4T1.2 cells. (E) No detectable change in the expression of Kdm3a and the selected tumor-promoting genes by the overexpression of Sox2 and Klf4 in 4T1.2 cells.

FIG. 22 illustrates prediction of the tumor suppressors in CM by mass spectrometry-based whole-genome proteomics. The single and double asterisks indicate p<0.05 and 0.01, respectively. CN=control, si=siRNA, and CM=conditioned medium. (A) Summary list of the potential tumor suppressors by mass spectrometry-based whole-genome proteomics. (B) Enolase 1 (Eno1), Hsp90ab1, Eef2, and Vcl as 4 tumor suppressor candidates based on MTT-based viability. (C) Upregulation of Eno1, Hsp90ab1, Eef2, VCL, p53, and Trail in 4T1.2 cell-derived CM with the overexpression of Oct4, c-Myc, and the treatment with OAC2. The overexpression of Sox2 and Klf4 did not alter their levels. (D-F) Elevation of MTT-based cell viability and Tranwell invasion of 4T1.2 tumor cells by CM, which was derived from 4T1.2 cells treated with siRNAs specific for Eno1, Hsp90ab1, Eef2, and VCL.

FIG. 23 illustrates contrasting tumor-promoting effects by the overexpression of Eno1, Eef2, and VCL in 4T1.2 mammary tumor cells, and tumor-suppressing effects by the administration of their recombinant proteins. The double asterisk indicates p<0.01. CN=control, Eno1=enolase 1, VCL=vinculin, and Hsp=Hsp90ab1. (A-C) Elevation in EdU-based proliferation, TRANSWELL® invasion, and the upregulation of Lrp5, MMP9, Runx2, TGFβ, and Snail by the overexpression of Eno1, Eef2, and VCL in 4T1.2 tumor cells. (D) Decrease in EdU-based proliferation by the administration of Eno1, Eef2, and VCL recombinant proteins. (E) Reduction in TRANSWELL® invasion by the administration of Eno1, Eef2, and VCL recombinant proteins. (F) Downregulation of Lrp5, MMP9, Runx2, TGFβ, and Snail in 4T1.2 tumor cells by the administration of Eno1, Eef2, and VCL recombinant proteins. (G) Reduction in TRANSWELL® invasion by Hsp90ab1 recombinant protein. (H) Reduction in the levels of Lrp5, MMP9, Runx2, TGFβ, and Snail by Hsp90ab1 recombinant protein.

FIG. 24 illustrates tumor selectivity and tumor-suppressing capability of Hsp90ab1, Eno1, Eef2, and VCL in 4T1.2 mammary tumor cells, and the suppression of the development of osteoclasts. CN=control, Hsp=Hsp90ab1, Eno1=enolase 1, VCL=vinculin, and CM=conditioned medium. The double asterisk indicates p<0.01. (A) Tumor selectivity from the MTT-based viability of tumor cells (4T1.2 mammary tumor cells, EO771 mammary tumor cells, and MDA-MB-231 breast cancer cells) and non-tumor cells (adipose cells, MLO-A5 osteocytes, and MSCs). Tumor selectivity is defined as a ratio of (MTT-based reduction in tumor cells) to (MTT-based reduction in non-tumor cells). (B) Reduction in Kdm3a in 4T1.2 cells in response to Eno1, Hsp90ab1, Eef2, and/or VCL recombinant proteins. (C) Elevation of Kdm3a in 4T1.2 cells by the overexpression of Eno1, Eef2, and VCL. (D) Suppression of RANKL-stimulated osteoclast development by Oct4-overexpressing CM. (E) Reduction in the levels of cathepsin K and NFATc1 in RANKL-stimulated osteoclasts by Oct4-overexpressing CM. (F) Regulatory mechanism for the tumor-suppressing action of tumor cell-derived iTS cells

FIG. 25 illustrates tumor-suppressing capability of OAC2-treated tumor cell-derived CM. The double asterisk indicates p<0.01. CN=control, and CM=conditioned medium. (A-C) Reduction in EdU-based proliferation, scratch-based migration, and TRANSWELL® invasion in 4T1.2 cells by OAC2-treated 4T1.2 tumor cell-derived CM. (D&E) Reduction in EdU-based proliferation and scratch-based migration in EO771 cells by OAC2-treated 4T1.2 tumor cell-derived CM. (F&G) Reduction in EdU-based proliferation and scratch-based migration in MDA-MB-231 breast cancer cells by OAC2-treated 4T1.2 tumor cell-derived CM.

FIG. 26 illustrates no significant effect by the direct administration of OAC2 to 4T1.2 tumor cells and mammary tumors. CN=control. (A) No detectable change in MTT-based viability of 4T1.2 cells by the administration of 1 to 5 μM OAC2. (B) No detectable change in the size of mammary tumors in BALB/c mice by the daily intraperitoneal injection of 10 mg/kg OAC2.

FIG. 27 illustrates reduction in the tumor-invaded area in the proximal tibia by CM derived from 4T1.2 cells, which was overexpressed with c-Myc and/or Oct4. CM=conditioned medium, and pl=placebo. The single and double asterisks indicate p<0.05 and 0.01, respectively.

FIG. 28 illustrates tumor-suppressing capability of Oct4- and c-Myc-overexpressing 4T1.2 tumor cell-derived CMs in EO771 mammary tumor cells and MDA-MB-231 breast cancer cells. The double asterisk indicates p<0.01. CN=control, Oct4=Oct4 plasmids, c-Myc=c-Myc plasmids, C+O=c-Myc and Oct4, and CM=conditioned medium. (A&B) Reduction in MTT-based viability and scratch-based migration of EO771 cells by Oct4- and c-Myc-overexpressing 4T1.2 tumor cell-derived CMs. (C&D) Reduction in MTT-based viability and scratch-based migration of MDA-MB-231 cells by Oct4- and c-Myc-overexpressing 4T1.2 tumor cell-derived CMs.

FIG. 29 illustrates no detectable effect on 4T1.2 tumor cells by the overexpression of Sox2 and Klf4. CN=control, Sox2=Sox2 plasmids, Klf4=Klf4 plasmids, and CM=conditioned medium. (A) Overexpression of Sox2 and Klf4, and no detectable effects on MTT-based viability in 4T1.2 cells by Sox2- or Klf4-overexpressing 4T1.2 tumor cell-derived CMs. (B-D) No detectable effects on scratch based migration in 4T1.2, E0771, and MDA-MD-231 cells, respectively, by Sox2- or Klf4-overexpressing 4T1.2 tumor cell-derived CMs.

FIG. 30 illustrates direct effect of the overexpression and silencing of Oct4 in 4T1.2 mammary tumor cells without employing CM. CN=control, Oct4=Oct4 plasmids, and siOct4=Oct4 siRNA. (A) Overexpression and silencing of Oct4 in 4T1.2 cells by plasmid transfection and RNA interference. (B) Elevation in the levels of Lrp5, MMP9, Runx2, TGFβ, and Snail by the overexpression of Oct4 in 4T1.2 cells. (C) Reduction in the levels of Lrp5, MMP9, Runx2, TGFβ, and Snail by the silencing of Oct4 in 4T1.2 cells.

FIG. 31 illustrates effects of silencing enolase 1 (Eno1), Hsp90ab1, Eef2, and vinculin in 4T1.2 cells. CN=control, CM=conditioned medium, and si=siRNA. (A) Stimulation of scratch-based migration of 4T1.2 tumor cells by 4T1.2 cell-derived CM, which was treated with siRNAs specific to Eno1, Hsp90ab1, Eef2, and VCL.(B) Elevation of the levels of Lrp5, MMP9, Runx2, TGFβ, and Snail in 4T1.2 cells in response to 4T1.2 cell-derived CM, treated with enolase 1 siRNA, Hsp90ab1 siRNA, Eef2 siRNA, and vinculin siRNA, respectively. (C) Downregulation of p53 and Trail in 4T1.2 cell-derived CM, treated with enolase 1 siRNA, Hsp90ab1 siRNA, Eef2 siRNA, and vinculin siRNA.

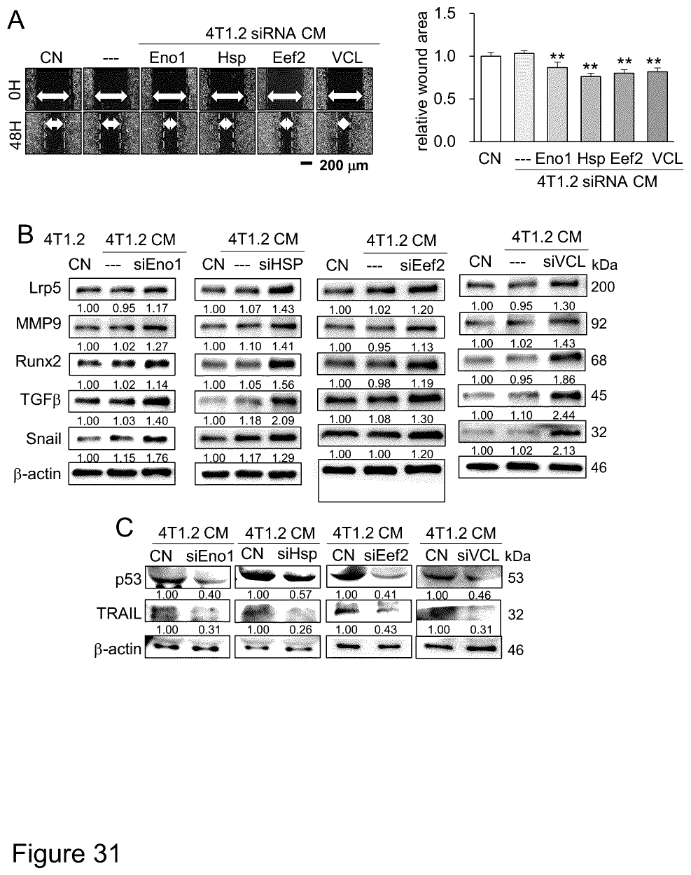

### DETAILED DESCRIPTION

The subject matter described herein relates to a seminal, far-reaching discovery that culturing cancerous mammalian cells that have been pre-activated to express tumorigenic genes generates a tumor-suppressing conditioned medium.

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

“Bone mineral density” refers to the inorganic mineral content in bone.

“Bone volume ratio” refers to the ratio of the segmented bone volume to the total volume.

“Cell growth signaling pathway activator” refers to any substance that enhances, or promotes or activates non-cancerous or cancerous mammalian cell growth and/or cell proliferation activity and/or cell migration activity. Mammalian cell growth signaling pathways include, but are not limited to, highly conserved pathways such as the Wnt signaling pathway, the PI3K signaling pathway, the Fibroblast Growth Factor (FGF) signaling pathway, and the Notch signaling pathway. Cell growth signaling pathway activators useful in the embodiments include small molecules, proteins, fusion proteins, and/or nucleic acids. In embodiments, cell growth signaling pathways include, but are not limited to the Wnt signaling pathway, the OCT3/4 signaling pathway, the PI3K signaling pathway, the Ras-ERK signaling pathway, the Fibroblast Growth Factor (FGF) signaling pathway, the Notch signaling pathway, the c-Myc signaling pathway, and the Epithelial Mesenchymal Transition (EMT) signaling pathway.

“Cell growth signaling pathway inhibitors” refers to any substance that diminishes or inhibits or inactivates non-cancerous or cancerous mammalian cell growth and/or cell proliferation activity. Cancerous cells are not treated with cell growth signaling pathway inhibitors in embodiments.

“Wnt signaling pathway” denotes a signaling pathway that may be divided in two pathways: the canonical Wnt/beta catenin signaling pathway and the “Wnt/PCP signaling pathway. Canonical Wnt/beta catenin signaling pathway” or Wnt/PCP signaling pathway denotes a network of proteins and other bioactive molecules (lipids, ions, sugars . . . ) best known for their roles in embryogenesis and cancer, but also involved in normal physiological processes in adult animals. The canonical Wnt/beta catenin signaling pathway is characterized by a Wnt dependant inhibition of glycogen synthase kinase 3β (GSK-3β), leading to a subsequent stabilization of β-catenin, which then translocates to the nucleus to act as a transcription factor. The Wnt/PCP signaling pathway does not involve GSK-30 or β-catenin, and comprises several signaling branches including Calcium dependant signaling, Planar Cell Polarity (PCP) molecules, small GTPases and C-Jun N-terminal kinases (JNK) signaling. These pathways are well known to those skilled in the art.

“Wnt signaling pathway activator” refers to a substance that enhances or promotes or activates a Wnt signaling activity. For example, for the canonical Wnt/β-catenin signaling pathway, this activity can be measured by Wnt reporter activity using established multimers of LEF/TCF binding sites reporters, and/or inhibition of GSK-30, and/or activation of canonical Wnt target genes such as T, Tbx6, Msgn1, or Axin2. An activation of a Wnt signaling activity may therefore be assessed as being an increase of a Wnt of Msgn1 reporter activity as identified above. The increase may be of at least 1%, 5% 10%, 15%, 20%, 25%, 30%, 35%, 40%, 45%, 50%, 55%, 60%, 65%, 70%, 75%, 80%, 85%, 90%, 95%, 100% or more. Wnt signaling pathway activators are known to those skilled in the art. Small molecule Wnt signaling pathway activators include, but are not limited to, BML-284, CHIR99021, and Wnt pathway activator 1.

“Epithelial-mesenchymal transition (EMT) pathway” refers to signaling pathways that relate to biologic processes that allow a normal or cancer cell to undergo multiple biochemical changes, thereby enabling it to assume a mesenchymal cell phenotype, e.g., enhanced migratory capacity, invasiveness, elevated resistance to apoptosis, and greatly increased production of ECM components. Classes of molecules that change in expression, distribution, and/or function during EMT, and that are causally involved, include growth factors (e.g., transforming growth factor-β (TGF-β), Wnts), EGF, HGF, transcription factors (e.g., Snail, SMAD, LEF, and nuclear β-catenin), molecules of the cell-to-cell adhesion axis (cadherins, catenins), cytoskeletal modulators (Rho family), and extracellular proteases (matrix metalloproteinases, plasminogen activators).

“EMT pathway activator” refers to a substance that enhances or promotes or activates a EMT pathway or activity.

“PI3K/Akt signaling pathway activator” refers to a substance that enhances or promotes or activates a PI3K/Akt signaling activity. Small molecule PI3K/Akt signaling pathway activators include, but are not limited to YS-49 and SC79.

“Notch signaling pathway activator” refers to a substance that enhances or promotes or activates a Notch signaling activity. Small molecule Notch signaling pathway activators include, but are not limited to, resveratrol. Small molecule FGF signaling pathway activators, small molecule OCT3/4 signaling pathway activators, small molecule c-Myc signaling pathway activators, small molecule Ras-ERK signaling pathway activators, and small molecule EMT signaling pathway activators are known to those skilled in the art.

Small molecule Oct4 signaling pathway activators include, but are not limited to Oct4 activating compound 2 (OAC2).

“Cancer” or “tumor” or “cancerous” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. “Cancer” refers to all types of cancer or neoplasm or malignant tumors found in humans, including, but not limited to: leukemias, lymphomas, melanomas, carcinomas and sarcomas. “Cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, or breast invasive carcinoma. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to metastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body that is different from the primary site. In embodiments disclosed herein, treatment is directed to the site of the cancer, not type of cancer, so that a cancer of any type that is situated in the lung, for example, would be treated on the basis of this localization in the lung.

For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers and mammalian cancerous cells that are relevant in some embodiments are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

“Triple negative breast cancer (TNBC)” refers to any breast cancer that does not express the genes for estrogen receptor (ER), progesterone receptor (PR) and Her2/neu. The term includes primary epithelial TNBCs, as well as TNBC that involved with other tumors. The cancer can include a triple negative carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. TNBC can also include any stage of triple negative breast cancer, and can include breast neoplasms having histologic and ultrastructual heterogeneity (e.g., mixed cell types).

“Cell” refers to the basic structural and functional unit of a living organism. In higher organisms, e.g., animals, cells having similar structure and function generally aggregate into “tissues” that perform particular functions. Thus, a tissue includes a collection of similar cells and surrounding intercellular substances, e.g., epithelial tissue, connective tissue, muscle, nerve. An “organ” is a fully differentiated structural and functional unit in a higher organism that may be composed of different types of tissues and is specialized for some particular function, e.g., kidney, heart, brain, liver, etc. Accordingly, by “specific organ, tissue, or cell” is meant herein to include any particular organ, and to include the cells and tissues found in that organ.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

“Cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

“Combination therapy” includes the administration of the subject compositions/proteins in further combination with other biologically active ingredients (such as, but not limited to, a second and different anti-cancer agent) and non-drug therapies (such as, but not limited to, surgery or radiation treatment). For instance, the compositions/proteins of some embodiments can be used in combination with other pharmaceutically active compounds, for instance compounds that are able to enhance the effect of the compositions/proteins of some embodiments. The compositions/proteins of some embodiments can be administered simultaneously (as a single preparation or separate preparation) or sequentially to the other drug therapy. In general, a combination therapy envisions administration of a conditioned media/protein and one or more drugs during a single cycle or course of therapy. In an embodiment, the administered biologically active ingredient is not a cell-growth signaling pathway activator.

“Conditioned medium” refers to a liquid nutrient medium that has been in contact with and exposed to cultured cancerous mammalian cells, where the mammalian cancerous cells produce metabolites, peptides, and proteins that enter the media, thus bestowing upon the media a therapeutic activity.

“Disease-free survival” refers to living free of the cancer being monitored. For example, if differential gene expression is used to diagnose or monitor breast cancer, disease-free survival would mean free from detectable breast cancer.

“Event-free survival” refers to living without the occurrence of a particular group of defined events (for example progression of cancer) after a particular action (e.g., treatment).

“Mammalian cell culture medium” and “culture medium” (or simply “medium”) refer to a nutrient solution used for growing mammalian cells that typically provides at least one component from one or more of the following categories: (1) salts (e.g., sodium, potassium, magnesium, calcium, etc.) contributing to the osmolality of the medium; (2) an energy source, usually in the form of a carbohydrate such as glucose; (3) all essential amino acids, and usually the basic set of twenty amino acids; (4) vitamins and/or other organic compounds required at low concentrations; and (5) trace elements, where trace elements are defined as inorganic compounds that are typically required at very low concentrations, usually in the micromolar range. The nutrient solution may optionally be supplemented with one or more of the components from any of the following categories: (a) animal serum; (b) hormones and other growth factors such as, for example, insulin, transferrin, and epidermal growth factor; and (c) hydrolysates of plant, yeast, and/or tissues, including protein hydrolysates thereof. Selection of the most appropriate culture medium is within the skill of those in the art.

“Fusion molecule” and “fusion protein” refer interchangeably to a biologically active polypeptide and an effector molecule covalently linked (i.e., fused) by recombinant, chemical or other suitable method. If desired, the fusion molecule can be fused at one or several sites through a peptide linker sequence. Alternatively, the peptide linker may be used to assist in construction of the fusion molecule. In embodiments, fusion molecules are fusion proteins. Generally fusion molecules also can be comprised of conjugate molecules.

“Increased” and grammatical equivalents (including “higher,” “bigger,” etc.) when in reference to the expression of any characteristic in a first subject relative to a second subject, mean that the quantity and/or magnitude of the characteristic in the first subject is lower than in the second subject by any amount that is recognized as clinically relevant by any medically trained personnel. In one embodiment, the quantity and/or magnitude of the characteristic in the first subject is at least 10% greater than, at least 25% greater than, at least 50% greater than, at least 75% greater than, and/or at least 90% greater than the quantity and/or magnitude of the characteristic in the second subject. In embodiments, either the first or second subject may be treated and the other of the first or second subject may be untreated. In embodiments, the first subject is untreated and the second subject is treated.

“Likelihood of reappearance” refers to the probability of tumor reappearance or metastasis in a subject subsequent to diagnosis of cancer.

“Likelihood of recovery” refers to the probability of disappearance of tumor or lack of tumor reappearance resulting in the recovery of the subject subsequent to diagnosis of cancer.

“Metastasis” is well known to one of skill in the art and refers to the growth of a cancerous tumor in an organ or body part, which is not directly connected to the organ of the original cancerous tumor.

“Nucleic acids” and “nucleic acid sequences” refer to oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs), nucleic acids, i.e., oligonucleotides, containing known analogues of natural nucleotides.

“Non-cancerous cells” refers to normal cells. Normal cells can be readily distinguished from primary cancer cells and metastatic cancer cells by well-established techniques, particularly histological examination.

“Selective toxicity” is the propensity of an anti-tumor agent or conditioned medium to affect tumor cells in preference to other healthy cells. In some embodiments, the pharmaceutical compositions and conditioned media are selectively toxic towards tumor cells. In other embodiments, the pharmaceutical compositions and conditioned media are not selectively toxic towards tumor cells and affect cancer cells and healthy cells.

“Overall survival” refers to the fate of a subject after diagnosis, despite the possibility that the cause of death in a subject is not directly due to the effects of the cancer.

“Pharmaceutically acceptable” and “pharmacologically acceptable” refer to compounds and compositions that do not produce adverse, allergic, or other untoward reactions when administered to an animal or a human.

In embodiments, the compositions may be administered to patients in a pharmaceutical composition comprising the conditioned medium (CM) along with a pharmaceutically acceptable carrier. The carrier may be any solvent, diluent, liquid or solid vehicle that is pharmaceutically acceptable and typically used in formulating compositions. Guidance concerning the making of pharmaceutical formulations can be obtained from standard works in the art (see, e.g., Remington's Pharmaceutical Sciences, 16th edition, E. W. Martin, Easton, Pa. (1980)). In addition, pharmaceutical compositions may contain any of the excipients that are commonly used in the art. Examples of carriers or excipients that may be present include, but are not limited to, sugars (e.g., lactose, glucose and sucrose); starches, such as corn starch or potato starch; cellulose and its derivatives (e.g., sodium carboxymethyl cellulose, ethyl cellulose, or cellulose acetate); malt; gelatin; oils (e.g., peanut oil, cottonseed oil, safflower oil, sesame oil, olive oil, corn oil, or soybean oil); glycols; buffering agents; saline; Ringer's solution; alcohols; lubricants; coloring agents; dispersing agents; preservatives; or antioxidants.

“Pharmaceutically acceptable salt” refers to pharmaceutically acceptable organic or inorganic salts of a compound of an embodiment. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Polypeptide” and “protein” refer interchangeably to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition. In various embodiments the polypeptides can have at least 10 amino acids or at least 25, or at least 50 or at least 75 or at least 100 or at least 125 or at least 150 or at least 175 or at least 200 amino acids.

“Progression-free survival” is well known to one of skill in the art and refers to the length of time during and after treatment in which a subject is living with a cancer that does not get worse, and can be used in a clinical study or trial to help find out how well a treatment is working.

“Reduced” and grammatical equivalents (including “lower,” “smaller,” etc.) when in reference to the expression of any characteristic in a first subject relative to a second subject, mean that the quantity and/or magnitude of the characteristic in the first subject is lower than in the second subject by any amount that is recognized as clinically relevant by any medically trained personnel. In one embodiment, the quantity and/or magnitude of the characteristic in the first subject is at least 10% lower than, at least 25% lower than, at least 50% lower than, at least 75% lower than, and/or at least 90% lower than the quantity and/or magnitude of the characteristic in the second subject. In embodiments, either the first or second subject may be treated and the other of the first or second subject may be untreated. In embodiments, the first subject is treated and the second subject is untreated.

“Small molecule” refers to a low molecular weight organic compound that may help regulate a biological process. Small molecules include any molecules with a molecular weight of about 2000 daltons or less, such as of about 500 to about 900 daltons or less. Small molecules can have a variety of biological functions, serving as cell signaling molecules, as drugs in medicine, and in many other roles. These compounds can be natural or artificial. Biopolymers such as nucleic acids and proteins, and polysaccharides (such as starch or cellulose) are not small molecules—though their constituent monomers, ribo- or deoxyribonucleotides, amino acids, and monosaccharides, respectively, are often considered small molecules. Small molecules include pharmaceutically acceptable salts of small molecules.

“Subject” refers to any mammal for whom diagnosis, treatment, or therapy is desired including mammals, e.g., humans, laboratory animals (e.g., primates, rats, mice, rabbits), livestock (e.g., cows, sheep, goats, pigs, turkeys, and chickens), household pets (e.g., dogs, cats, and rodents), and horses.

“Substantially homogenous” refers to a population of cells derived from the same mammalian organ or region of a mammalian organ wherein the majority between about 100% to about 70%; between about 100% to about 90% of the total number of cells have a specified characteristic of interest.

“Therapeutically effective amount” is that amount sufficient, at dosages and for periods of time necessary, to achieve a desired therapeutic result, such as for treatment of a disease (e.g. cancer), condition, or disorder, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject. One skilled in the art will recognize that the condition of the individual can be monitored throughout the course of therapy and that the effective amount of a compound or composition disclosed herein that is administered can be adjusted accordingly.

“Trabecular number” refers to the average number of trabeculae per unit length.

“Trabecular separation” refers to the mean distance between trabeculae.

“Treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer. “Treat” covers any treatment of a cancer in a mammal, and includes: (a) preventing the cancer from occurring in a subject which may be predisposed to the disease but has not yet been diagnosed as having it; (b) inhibiting the cancer, i.e., arresting its development; or (c) relieving the cancer, i.e., causing regression of the cancer. The therapeutic composition may be administered before, during or after the onset of cancer. The therapy may be administered during the symptomatic stage of the cancer, and in some cases after the symptomatic stage of the cancer.

“Tumor microenvironment” or “cancer microenvironment” refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the tumor microenvironment. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

Aspects of the present disclosure can be described as embodiments in any of the following enumerated clauses. It will be understood that any of the described embodiments can be used in connection with any other described embodiments to the extent that the embodiments do not contradict one another.

Clause. A pharmaceutical composition comprising a cell-free conditioned medium (CM) or extract or concentrate thereof obtained from a mammalian cell culture medium comprising a cultured substantially homogenous cancerous mammalian cell population where a portion of the cancerous mammalian cell population is contacted by a small molecule cell growth signaling pathway activator before being cultured in the cell culture medium.

Clause. A pharmaceutical composition according to the preceding clause, where the conditioned medium is concentrated.

Clause. A pharmaceutical composition according to any of the preceding clauses, where the a portion of the cancerous mammalian cell population is contacted by at least two small molecule cell growth signaling pathway activators before being cultured in the cell culture medium.

Clause. A pharmaceutical composition according to any of the preceding clauses, further comprising a pharmaceutically acceptable carrier.

Clause. A pharmaceutical composition according to any of the preceding clauses, where the conditioned medium further comprises a cancerous mammalian cell-secreted protein selected from the group consisting of heat shock protein 90 alpha family class B member 1 (Hsp90ab1), enolase 1 (Eno1), eukaryotic translation elongation factor 2 (Eef2), ubiquitin C (Ubc), and vinculin (VCL).

Clause. A pharmaceutical composition according to any of the preceding clauses, where the composition is enriched with a cancerous mammalian cell-secreted protein selected from the group consisting of heat shock protein 90 alpha family class B member 1 (Hsp90ab1), enolase 1 (Eno1), eukaryotic translation elongation factor 2 (Eef2), ubiquitin C (Ubc), and vinculin (VCL).

Clause. A pharmaceutical composition according to any of the preceding clauses, further comprising a chemotherapeutic agent.

Clause. A pharmaceutical composition according to any of the preceding clauses, where the small molecule cell growth signaling pathway activator is a small molecule Wnt signaling pathway activator.

Clause. A pharmaceutical composition according to any of the preceding clauses, where the small molecule Wnt signaling pathway activator is BML-284, or a pharmaceutically acceptable salt thereof.

Clause. A pharmaceutical composition according to any of the preceding clauses, where the cancerous mammalian cells are cancerous mammalian bone cells.

Clause. A pharmaceutical composition according to any of the preceding clauses, where the cancerous mammalian bone cells are osteocytes.

Clause. A pharmaceutical composition according to any of the preceding clauses, where the cancerous mammalian cells are cancerous mammalian bone cells isolated from bone marrow.

Clause. A kit comprising: a) a pharmaceutical composition according to any of the preceding clauses; b) a container; c) a label; and d) instructions that provide methods for administering the composition.

Clause. A kit according to the preceding clause, where the pharmaceutical composition further comprises at least one preservative.

Clause. A method to treat a cancer in a subject in need thereof, the method comprising: administering to the subject in need thereof a therapeutically effective amount of a cell-free conditioned medium (CM) or extract or concentrate thereof obtained from a mammalian cell culture medium comprising a cultured substantially homogenous cancerous mammalian cell population where a portion of the cancerous mammalian cell population is contacted by a small molecule cell growth signaling pathway activator before being cultured in the cell culture medium.

Clause. A method according to the preceding clause, where the subject is a human.

Clause. A method according to any of the preceding clauses, where the treated cancer is a metastatic cancer and the cultured substantially homogenous cancerous mammalian cell population is derived from a same organ or tissue as the organ or site to be treated.

Clause. A method according to any of the preceding clause where the treated cancer is a metastatic cancer and the cultured substantially homogenous cancerous mammalian cell population is derived from the same subject as the subject to be treated.

Clause. A method according to any of the preceding clauses, where the treated cancer is a metastatic cancer selected from the group consisting of metastatic bone cancer, metastatic liver cancer, metastatic lung cancer, and metastatic brain cancer.

Clause. A method according to any of the preceding clauses, where the treated cancer is a metastatic bone cancer.

Clause. A method according to any of the preceding clauses, where the treated cancer is a primary cancer.

Clause. A method according to any of the preceding clauses, where the treated cancer is a primary cancer selected from the group consisting of breast cancer, lung cancer, colorectal cancer, prostate cancer, skin cancer, and pancreatic cancer.

Clause. A method according to any of the preceding clauses, where the treated primary cancer is breast cancer selected from the group consisting of Estrogen Receptor (ER)-positive breast cancer, Estrogen Receptor (ER)-negative breast cancer, and triple-negative breast cancer.

Clause. A method according to any of the preceding clauses, where the cancerous mammalian cell population is cancerous mammalian bone cells selected from the group consisting of osteocytes, bone marrow-derived mesenchymal stem cells, and osteoblasts.

Clause. A method according to any of the preceding clauses, where the small molecule cell growth signaling pathway activator is selected from the group consisting of a small molecule Wnt signaling pathway activator, a small molecule PI3K signaling pathway activator, a small molecule FGF signaling pathway activator and a small molecule Notch signaling pathway activator.

Clause. A method according to any of the preceding clauses, where the small molecule cell growth signaling pathway activator is a small molecule Wnt signaling pathway activator.

Clause. A method according to any of the preceding clauses, where the small molecule Wnt signaling pathway activator is BML-284, or a pharmaceutically acceptable salt thereof.

Clause. A method according to any of the preceding clauses, where the cancer is metastatic bone cancer and the treatment reduces cancer-induced osteolysis.

Clause. A method according to any of the preceding clauses, where the cancer is breast cancer and the treatment reduces mammary tumor size.

Clause. A method according to any of the preceding clauses, where the cancer is metastatic bone cancer and the treatment increases bone volume ratio.

Clause. A method according to any of the preceding clauses, where the cancer is metastatic bone cancer and the treatment increases bone mineral density.

Clause. A method according to any of the preceding clauses, where the cancer is metastatic bone cancer and the treatment increases trabecular number.

Clause. A method according to any of the preceding clauses, where the cancer is metastatic bone cancer and the treatment reduces trabecular separation.

Clause. A method according to any of the preceding clauses, where the cancer is metastatic bone cancer and the treatment reduced osteoclastogenesis.

Clause. A method according to any of the preceding clauses, where the conditioned medium is in a pharmaceutical form suitable for systemic administration.

Clause. A method according to any of the preceding clauses, where the conditioned medium is in a pharmaceutical form suitable for local administration.

Clause. A method according to any of the preceding clauses, where the conditioned medium is in a pharmaceutical form suitable for administration by injection.

Clause. A method according to any of the preceding clauses, further comprising administering a chemotherapeutic agent.

Clause. A method according to any of the preceding clauses where apoptosis is not induced in normal cells.

Clause. A method according to any of the preceding clauses, where the method results in at least one activity selected from the group consisting of upregulating a tumor-suppressing gene in a cancer cell, downregulating a tumor-promoting gene in a cancer cell, inhibiting cancer cell invasion, inhibiting cancer cell growth and inhibiting cancer cell recurrence.

Clause. A method according to any of the preceding clauses, where the target cancer is a therapy-resistant cancer

Clause. A process to produce a conditioned medium (CM), the process comprising: contacting cancerous mammalian cells by a small molecule cell growth signaling pathway activator to generate pre-treated cancerous mammalian cells; culturing the pre-treated cancerous mammalian cells in a mammalian cell culture medium for a period of time sufficient to condition the medium; removing the pre-treated cancerous mammalian cells from the culture medium; and, collecting the conditioned medium.

Clause. A process according to the preceding clause, further comprising centrifuging the conditioned medium to remove exosomes.

Clause. A process according to any of the preceding clauses, where the mammalian cell culture medium is serum-free.

Clause. A process according to any of the preceding clauses, where the pre-treated cancerous mammalian cells are cultured for a time period from about 1 hour to about 24 hours.

Clause. A process according to any of the preceding clauses, further comprising filtering the collected conditioned medium.

Clause. A process according to any of the preceding clauses, further comprising ultra-filtering the collected conditioned medium.

Clause. A process according to any of the preceding clauses, further comprising concentrating the collected conditioned medium.

Clause. A process according to any of the preceding clauses, further comprising purifying the collected conditioned medium.

Clause. A method to identify an anti-tumor property in a conditioned medium (CM), the method comprising: contacting cancerous mammalian cells by a small molecule cell growth signaling pathway activator to generate pre-treated cancerous mammalian cells; culturing the pre-treated cancerous mammalian cells in a mammalian cell culture medium to condition the medium; removing the pre-treated cancerous mammalian cells from the culture medium; collecting the conditioned medium; and, assaying the conditioned medium for an anti-tumor property.

Clause. A method to treat cancer in a subject in need thereof, comprising administering to the subject an effective amount of heat shock protein 90 alpha family class B member 1 (Hsp90ab1).

Clause. A method to treat cancer in a subject in need thereof, comprising administering to the subject an effective amount of enolase 1 (Eno1).

Clause. A method to treat cancer in a subject in need thereof, comprising administering to the subject an effective amount of eukaryotic translation elongation factor 2 (Eef2).

Clause. A method to treat cancer in a subject in need thereof, comprising administering to the subject an effective amount of ubiquitin C (Ubc).

Clause. A method to treat cancer in a subject in need thereof, comprising administering to the subject an effective amount of vinculin (VCL).

### EXAMPLES

Examples related to the present disclosure are described below. In some embodiments, alternative techniques can be used. The examples are intended to be illustrative and are not limiting or restrictive of the scope of the claimed subject matter. Other variations or embodiments will be apparent to a person of ordinary skill in the art from the above-description.

### Example 1

Cell culture and agents. MLO-A5 osteocyte-like cells (obtained from Dr. L. Bonewald at Indiana University, IN, USA) and RAW 264.7 pre-osteoclast cells (ATCC, Manassas, VA, USA) were cultured in a-MEM. EO771 mouse mammary tumor cells (Corning, Inc., Corning, NY, USA), 4T1.2 mouse mammary tumor cells (obtained from Dr. R. Anderson at Peter MacCallum Cancer Institute, Melbourne, Australia), Panc-1 human pancreatic cancer cells (ATCC), and MCF-7 human estrogen receptor (ER)-positive breast cancer cells (ATCC) were cultured in Dulbecco's Modified Eagle Medium (“DMEM”). TRAMP-C2ras prostate tumor cells (ATCC) were cultured in DMEM/F-12. MDA-MB-231 human estrogen receptor (ER)-negative breast cancer cells and PC-3 human prostate cancer cells (ATCC) were cultured in Roswell Park Memorial Institute 1640 Medium (“RPMI-1640”). For the tumor cells, the culture media were supplemented with 10% fetal bovine serum (“FBS”) and antibiotics (50 units/ml penicillin, and 50 μg/ml streptomycin; Life Technologies, Grand Island, NY, USA). For MLO-A5 cells, the media contained 5% FBS, 5% fetal calf serum, and antibiotics. Cells were maintained at 37° C. and 5% CO₂.

Using 2×10⁶ different types of cells, β-catenin (40 ng/μ1) was transfected to generate induced tissue-specific stem (“iTS”) cells. Conditioned medium (CM, 9 ml culture medium) was prepared with antibiotics and a fraction of FBS including factors of 3 kDa or smaller. After one day of incubation, the medium was ultra-centrifuged to remove exosomes and condensed 10-fold by filtering (Amicon, Sigma, Saint Louis, MO, USA) with a cut-off molecular weight at 3 kDa. Proteins from CM-treated cells were harvested 24 h after the onset of incubation. Enolase 1 (500 ng/ml), and ubiquitin C (500 ng/ml, Mybiosource, San Diego, California, USA) recombinant proteins were given to EO771 cells, and cells were incubated for 24 h. A pharmacological inhibitor of enolase 1 (ENOBLOCK™—AP-III-a4, Mybiosource) and an inhibitor of E3 ubiquitin ligase (Pomalidomide, Mybiosource) were applied to the cells for 24 h.

EdU assays. Two thousand cells were seeded in 96-well plates on day 1, CM was added on day 2, and cellular proliferation was examined using a fluorescence-based cell proliferation kit (CLICK-IT™ EdU ALEXA FLUOR™ 488 Imaging Kit; Thermo-Fisher, Waltham, MA, USA). After fluorescent labeling, the number of fluorescently labeled cells were counted and the ratio to the total number of cells was determined.

Invasion assay. The invasion capacity of cancer cells was determined using a 24-well plate, TRANSWELL® chambers (Thermo Fisher Scientific, Waltham, MA, USA) with 8-mm pore size and MATRIGEL™. Cell invasion was measured in TRANSWELL® chambers with a coating including MATRIGEL™ (100 μg/ml). The cells in 200 μL serum-free DMEM were placed on the upper chamber and 800 μL iTS CM was added in the lower chamber. After 24 h, the cells that had invaded the lower side of the membrane were stained with Crystal Violet. At least five randomly chosen images were taken under a microscope, and the average number of stained cells that represented the relative invasion was determined.

Two-dimensional motility assay. A wound-healing scratch motility assay was performed to measure 2-dimensional cell motility. Cells were seeded in 12-well plates, after cell attachment, a plastic pipette tip was used to scratch a gap on the cell layer. Images of the cell-free scratch zone were obtained via an inverted microscope at 0 h, and the areas newly occupied with cells were determined 48 h after scratching. The areas were quantified with Image J (National Institutes of Health, Maryland, USA).

3D spheroid assay. The cells were cultured in separate wells of the U-bottom low-adhesion 96-well plate (S-Bio, Hudson, NH, USA). Spheroids were cultured in complete DMEM (10% FBS, 1% antibiotics). To evaluate the effect of iTS CM, the medium was replaced by CM, and spheroid images were captured after 48 h. Fluorescently labeled EO771 cells were prepared by culturing them with a green (#4705, Sartorius, Gottingen, Germany) or red fluorescent dye (#4706) for 20 min at 37° C. Cells were then harvested as a pellet by centrifuging at 1000 rpm for 5 min.

Western blot analysis. Cultured cells were lysed in a radio-immunoprecipitation assay buffer with protease inhibitors (Santa Cruz Biotechnology, Santa Cruz, CA, USA) and phosphatase inhibitors (Calbiochem, Billerica, MA, USA). After cell lysis, proteins were fractionated by 10-15% SDS gels and electro-transferred to polyvinylidene difluoride (“PVDF”) transfer membranes (Millipore, Billerica, MA, USA). After blocking 1 h with a blocking buffer (Bio-Rad), the membrane was incubated overnight with primary antibodies and then with secondary antibodies conjugated with horseradish peroxidase for 45 min (Cell Signaling, Danvers, MA, USA). Antibodies against β-catenin, cleaved-caspase 3, Runx2, Snail, Enolase-1 (Cell Signaling, Danvers, MA, USA), MMP9 (Santa Cruz, Dallas, TX, USA), TRAIL (Novus, Centennial, CO, USA), p53 (Invitrogen, Carlsbad, CA, USA), and β-actin as a control (Sigma, St. Louis, MO, USA), were employed. The level of proteins was determined using a SUPERSIGNAL™ west femto maximum sensitivity substrate (Thermo Scientific, Waltham, MA, USA), and a luminescent image analyzer (LAS-3000, Fuji Film, Tokyo, Japan) was used to quantify signal intensities.

Transfection. For overexpressing β-catenin, E0771, and other cells were transfected with plasmids (#31785, Addgene, Watertown, MA, USA), while blank plasmids (FLAG-HA-pcDNA3.1; Addgene) were used as a control. Cells were grown in a 10 cm-plate and transfected with β-catenin plasmids, or control plasmids using LIPOFECTAMINE®3000 (Thermo, L300015). The transfection reagents and DNA were mixed in two steps. In the first step, plasmids were diluted in 200 μL Opti-MEM and P3000 was added at the transfection-reagent/DNA ratio of 2 μl:1 μg. In the second step, 20 μL LIPOFECTAMINE®3000 was mixed with 200 μL Opti-MEM. These two mixtures were incubated at RT and the transfection was performed overnight. RNA interference with specific siRNAs was conducted to silence enolase 1, ubiquitin C, p53, and Trail, together with a nonspecific negative control siRNA (Silencer Select #1, Life Technologies; On-target Plus Non-targeting Pool, Dharmacon). Cells were transiently transfected with siRNA with LIPOFECTAMINE™ RNAiMAX (Life Technologies), and the medium was replaced by a regular culture medium after 24 h. The efficiency of silencing was assessed with immunoblotting 24 h after transfection.

Ex vivo tissue assay. The usage of human breast cancer and prostate cancer tissues was approved by the Indiana University Institutional Review Board. Two human breast cancer tissues (˜1 g each; estrogen receptor-positive and -negative) and prostate cancer tissue (˜1 g), received from Simon Cancer Center Tissue Procurement Core, were manually fragmented with a scalpel into small pieces (0.5˜0.8 mm in length). These pieces were grown in DMEM with 10% fetal bovine serum and antibiotics for a day. iTS cell-derived conditioned medium was then added for three additional days, and a change in the fragment size was determined. BML284 (10 μM) or β-catenin (40 ng/μ1) was applied to tissue fragments and CM was prepared as described above.

Animal models. The experimental procedures using animals were approved by the Indiana University Animal Care and Use Committee and were complied with the Guiding Principles in the Care and Use of Animals endorsed by the American Physiological Society. Mice were housed five per cage and provided with mouse chow and water ad libitum. In the mouse model of mammary tumors, C57BL/6 female mice (˜8 weeks, Envigo RMS, Inc., Indianapolis, IN, USA) were randomly assigned into five groups (14 mice per group). The placebo groups received cells transfected with a negative control vector, and the negative control group received cells transfected with β-catenin overexpression vector. The three treatment groups received β-catenin overexpressing iTS CM, BML-treated cells, and BML-treated iTS CM. Each group received a subcutaneous injection of EO771 cells (3.0×10⁵ cells in 50 μl PBS) to the mammary fat pad. For tibial osteolysis, C57BL/6 female mice (8 mice per group) received an intra-tibial injection of EO771 cells (2.5×10⁵ cells in 20 μl PBS) to the right tibia. The treatment group received iTS cell-derived CM as an injection into the intraperitoneal cavity. The animals were sacrificed on day 18, and the weight of each tumor was measured. To evaluate the effects of CMs for tumor invasion, an in vivo extravasation assay was conducted. EO771 cells were labeled with a green fluorescent dye and injected with and without iTS cell-derived CM via a lateral tail vein. Mice were sacrificed after 48 h for histological identification of extravascular tumor cells in the lung.

μCT imaging and histology. The tibia was harvested for μCT imaging using SKYSCAN™ 1172 (Bruker-MicroCT, Kontich, Belgium) and histology. The samples were analyzed in a blinded fashion. Using the manufacturer-provided software, CT scans were performed with a pixel size of 8.99 μm and the captured images were reconstructed (nRecon v1.6.9.18) and analyzed (CTan v1.13). In histology, H&E staining was conducted as described previously, and immunohistochemistry was performed using the procedure previously described.

Survival analysis. The pan-cancer survival analysis employed 9,880 primary tumor samples from 32 types of cancers from the UCSC Xena browser. The high expression group was defined to present the transcript level of MMP9, RUNX2, or Snail above the median value, while the low expression group below the median value. The Kaplan-Meier curve and log-rank test were used to evaluate survival probabilities with the survival package in R (v3.6.3).

Whole-genome proteomics analysis. Proteins in CM were analyzed in the DIONEX ULTIMATE™ 3000 RSLC nano system combined with the Q-exactive high-field hybrid quadrupole orbitrap mass spectrometer (Thermo Fisher Scientific). Proteins were first digested on-beads using trypsin/LysC as described previously except digestion was performed in 50 mM ammonium bicarbonate buffer instead of urea. Digested peptides were then desalted using mini spin C18 spin columns (The Nest Group, Southborough, MA, USA) and separated using a trap and 50-cm analytical columns (50, 52). Raw data were processed using MAXQUANT™ (v1.6.3.3) against the Uniprot mouse protein database at a 1% false discovery rate allowing up to 2 missed cleavages. MS/MS counts were used for relative protein quantitation. Proteins identified with at least 1 unique peptide and 2 MS/MS counts were considered for the final analysis.

Statistical analysis. For cell-based experiments, three or four independent experiments were conducted and data were expressed as mean±S.D. In animal experiments, the sample size in the mouse model was chosen to achieve a power of 80% with p<0.05. The primary experimental outcome was tumor weight for the mammary fat pad experiment and the bone volume ratio (BV/TV) for the tibia experiment. The secondary experimental outcome was tumor size for the mammary fat pad experiment and the trabecular number (Tb.n) for the tibia experiment. Statistical significance was evaluated using a one-way analysis of variance (ANOVA). Post hoc statistical comparisons with control groups were performed using Bonferroni correction with statistical significance at p<0.05. The single, double, and triple asterisks in the figures indicate p<0.05, 0.01, and 0.0001, respectively.

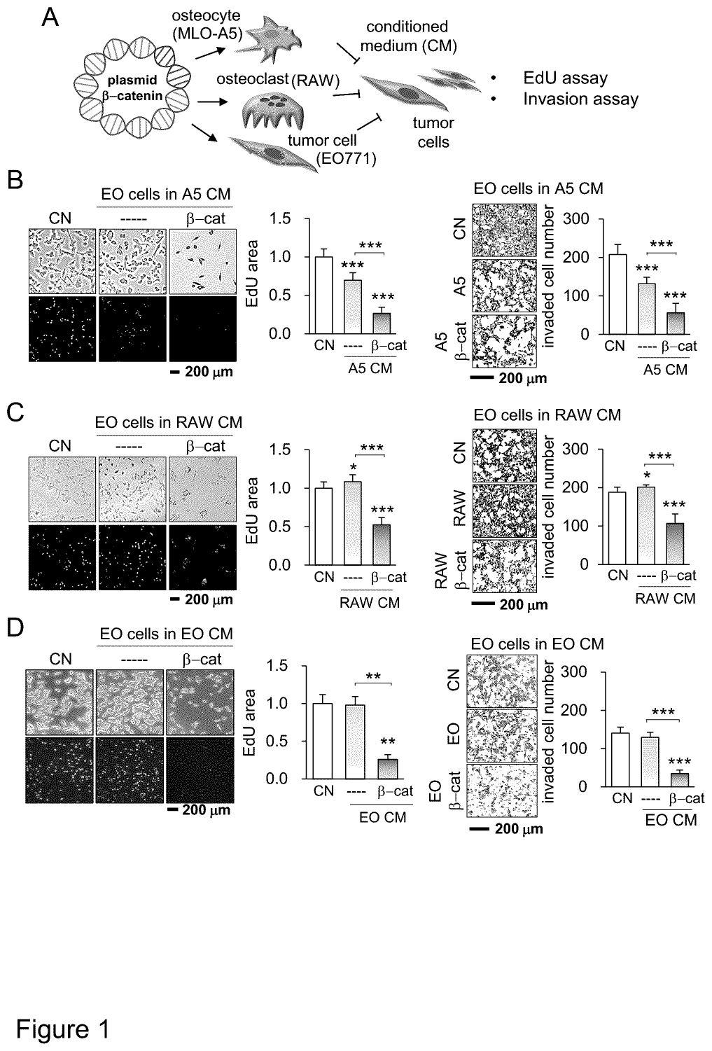

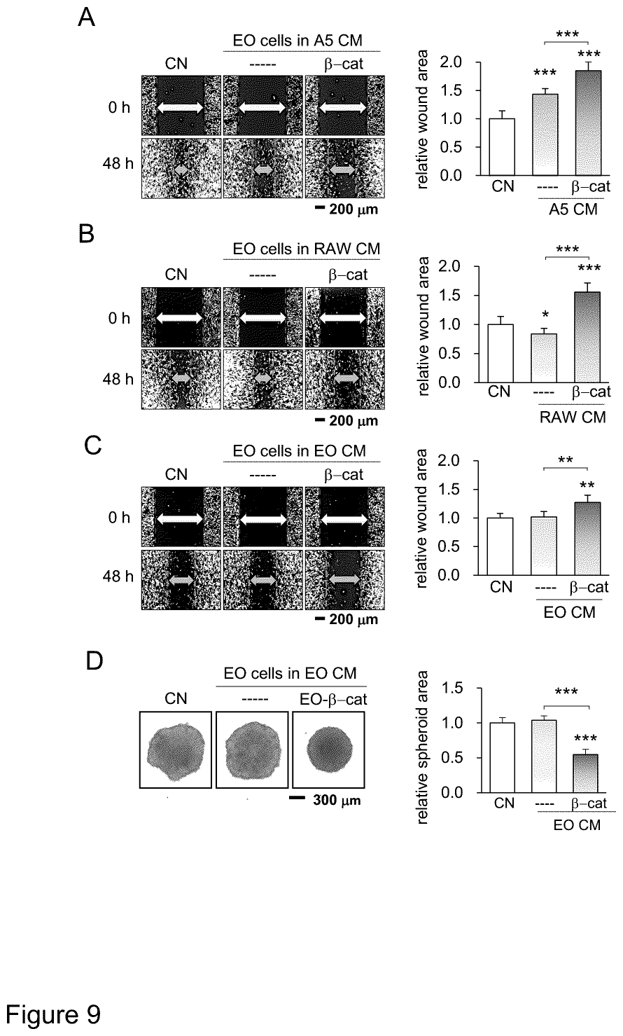

The overexpression of β-catenin generated iTS cells. To test the effect of the overexpression of β-catenin, constitutively active plasmids of β-catenin were transfected into MLO-A5 osteocytes, RAW264.7 osteoclasts, and EO771 mammary tumor cells (FIG. 1A). When EO771 cells were cultured in β-catenin-overexpressing cell-derived CMs, the EdU-based proliferation, scratch-based migration, TRANSWELL® invasion, and the growth of 3-dimensional spheroids of mammary tumor cells were significantly reduced, regardless of the source of CMs (FIGS. 1B-D; FIG. 9). The result provided the first evidence that the overexpression of β-catenin could generate iTS cells from osteocytes, osteoclasts, and mammary tumor cells.

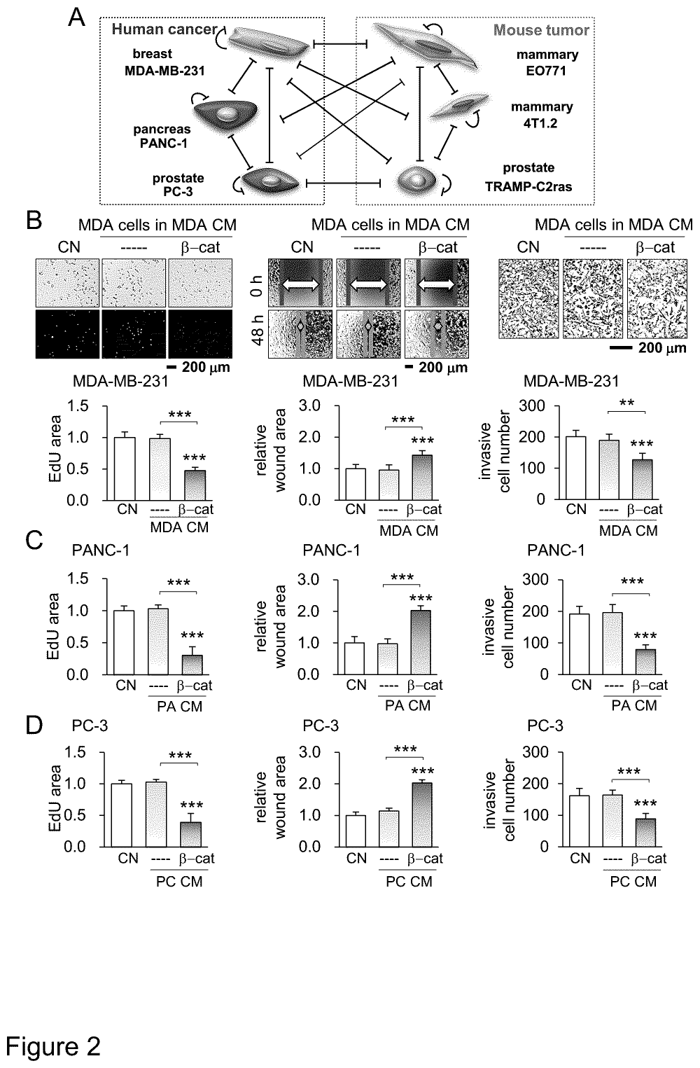

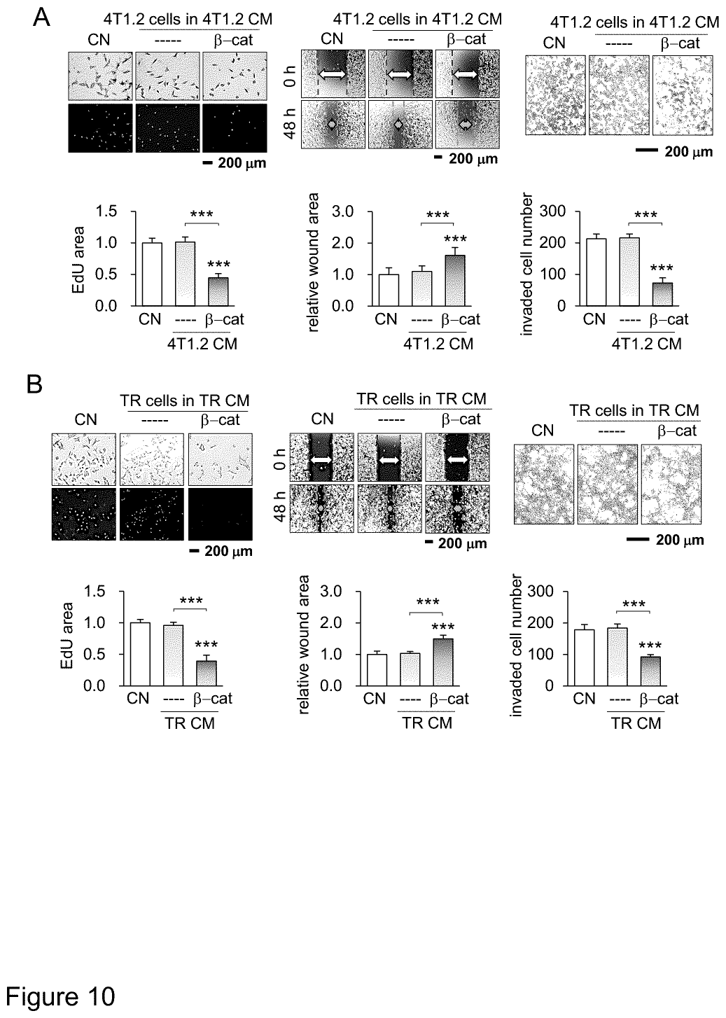

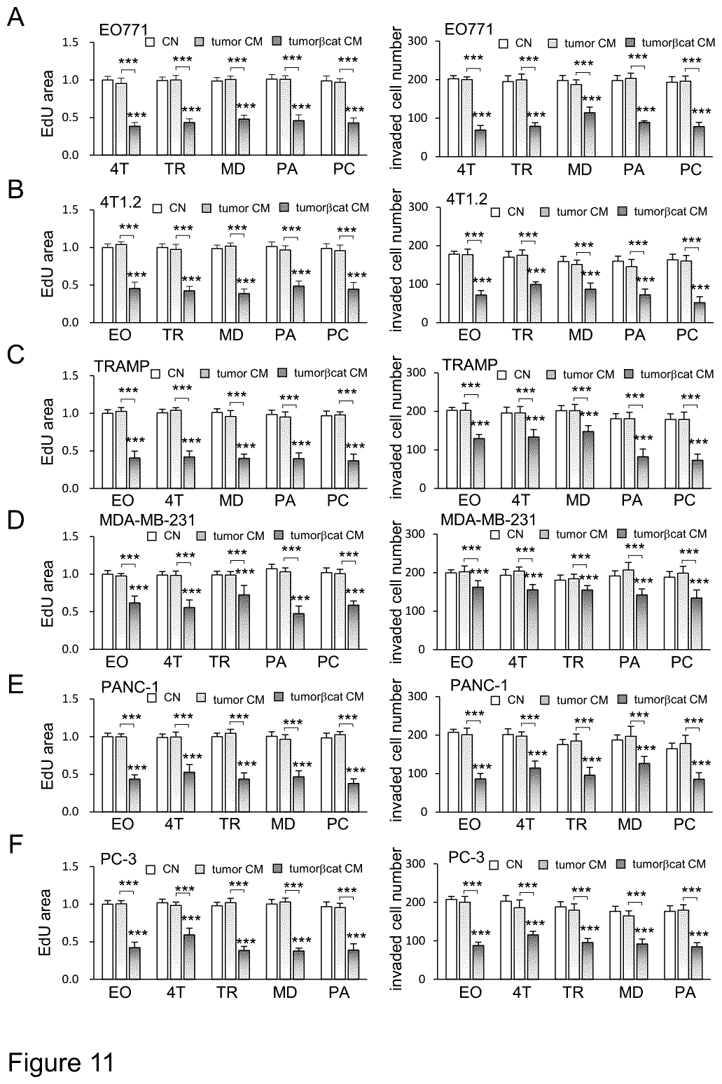

Cancer cells could become iTS cells. To further examine the possibility of generating iTS cells, β-catenin was overexpressed in human and mouse cancer cell lines originating from cancers in the breast, pancreas, and prostate (FIG. 2A). The result revealed that iTS cancer cell-derived CM was able to inhibit the proliferation and invasion of their starting cancer cells as well as other cancer cells. For instance, MDA-MB-231 breast cancer cell-derived CM inhibited EdU-based proliferation, scratch-based migration, and TRANSWELL® invasion of MDA-MB-231 breast cancer cells (FIG. 2B). The same responses were observed with PANC-1 pancreatic, PC-3 prostate, 4T1.2 mammary, and TRAMP prostate tumor cells (FIGS. 2C&D, FIG. 10). Each of the iTS CMs from the six selected cancer cell lines strikingly suppressed EdU-based proliferation, and TRANSWELL® invasion of the five non-self-cancer cells (FIG. 11). Collectively, iTS cells could be generated by the overexpression of β-catenin in non-tumor cells as well as tumor cells.

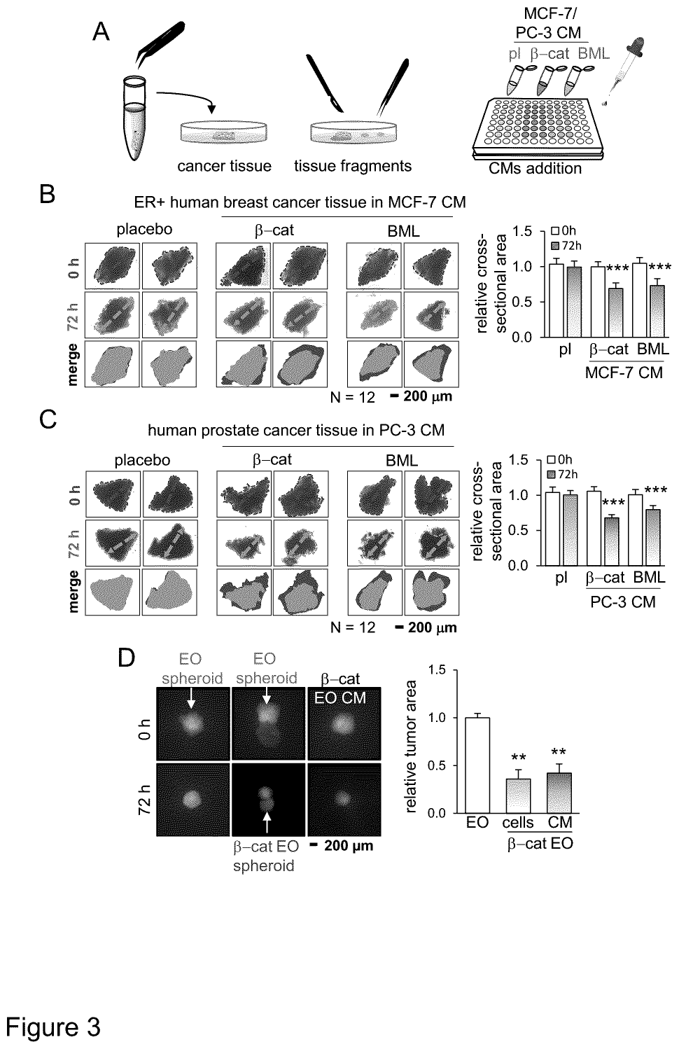

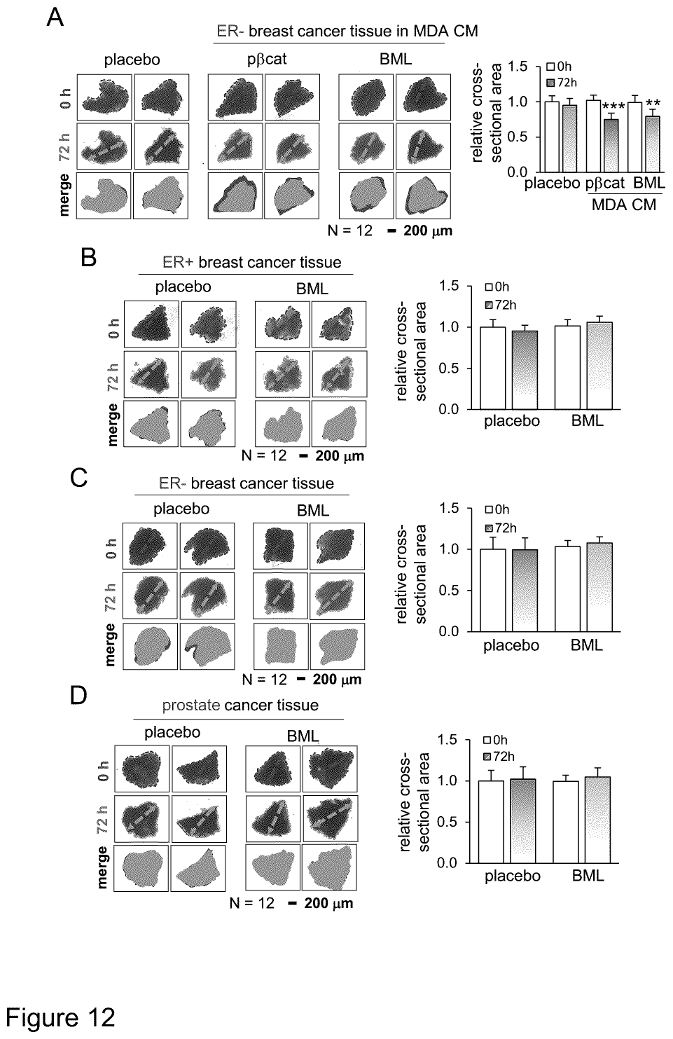

iTS cell-derived CM inhibited the growth of cancer tissue fragments. Having shown the anti-tumor capability of iTS CM, the efficacy in tumor suppression was next evaluated using 3 freshly isolated human cancer tissues from patients with breast cancer (estrogen receptor-positive and -negative) and prostate cancer. CM was prepared from the cancer cells by transfecting β-catenin plasmids or applying BML284 (FIG. 3A). Compared to the control CM that did not contain cell-originated factors, both CMs generated with β-catenin plasmids and BML284 significantly shrank the size of cancer tissue fragments in the ex vivo tissue assay (FIGS. 3B&C; FIG. 12A). By contrast, the direct addition of BML284 to the fragments modestly increased their size, although this effect did not reach statistical significance (FIGS. 12B-D). Thus, iTS CMs, prepared from the freshly isolated human breast and prostate cancer cells, were effective in suppressing the growth of tumor fragments. When a pair of tumor spheroids (red and green with and without β-catenin overexpression, respectively) was placed side by side, the red β-catenin-overexpressing spheroid inhibited the growth of the green spheroids. Similarly, CM from β-catenin-overexpressing cells shrank the green control spheroids (FIG. 3D).

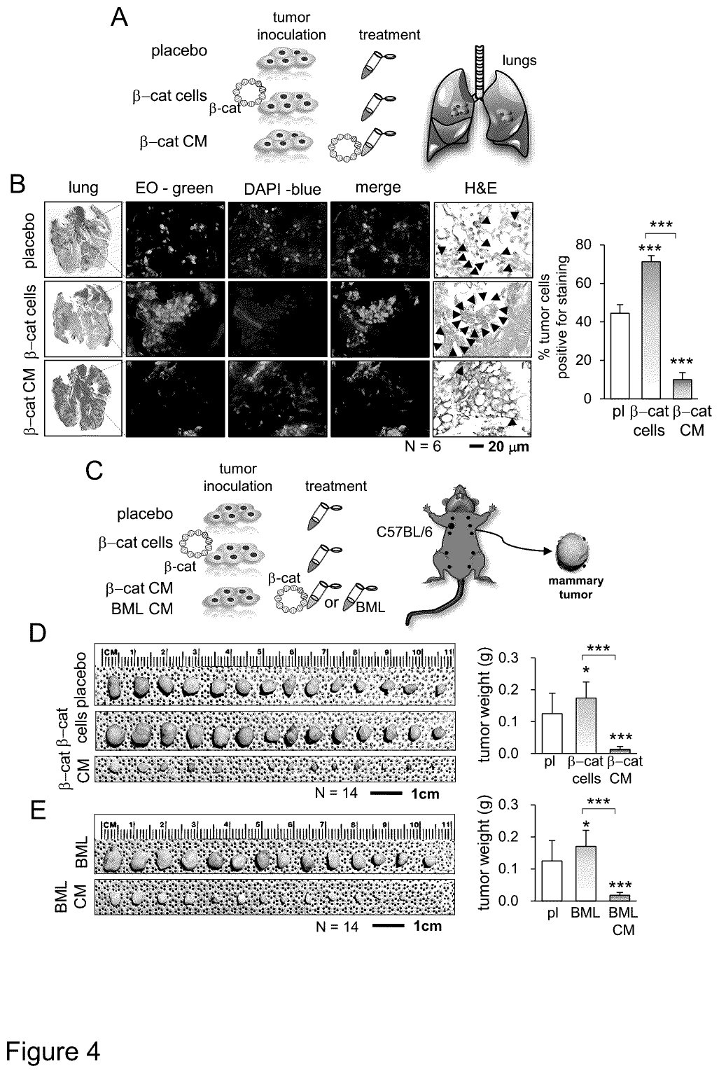

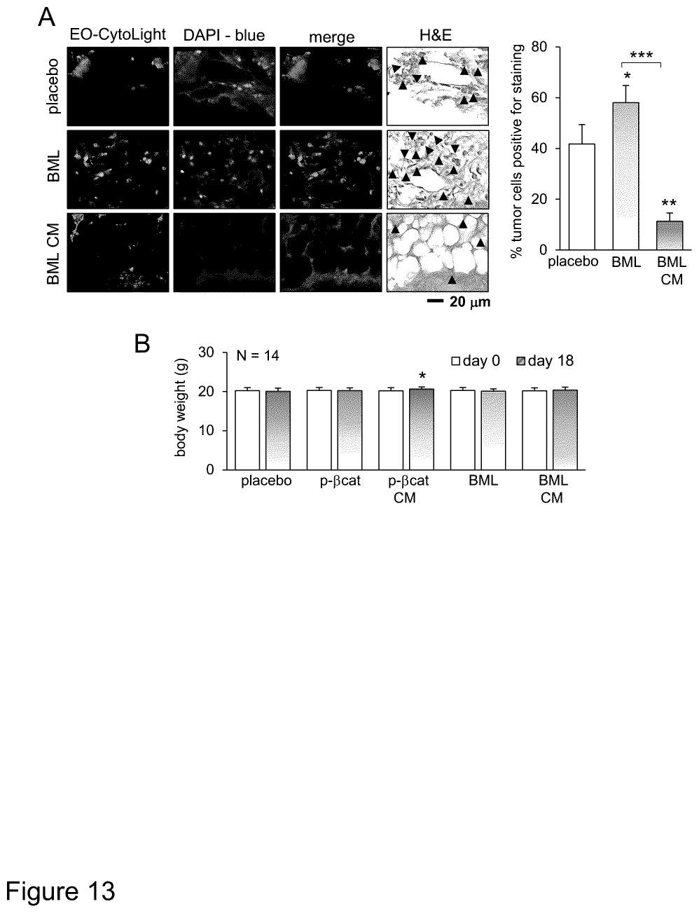

iTS CM inhibited the invasion and growth of mammary tumors. The efficacy of iTS CM was next examined in the mouse model. EO771 mammary tumor cells were intravenously injected into the tail vein in the extravasation assay or inoculated to the mammary fat pad in the mammary tumor assay. Compared to placebo mice that received control CM, two iTS CM groups (β-catenin-overexpression and BML284 pre-treatment), which received a daily intravenous injection of each of their CMs, markedly reduced the number of tumor cells in the lung (FIGS. 4A&B; FIG. 13A), as well as the size of mammary tumors (FIGS. 4C-E). By contrast, the direct inoculation of β-catenin-overexpressing EO771 tumor cells enlarged the tumor size in the mammary fat pad. The average body weight did not significantly change during the treatment (FIG. 13B).

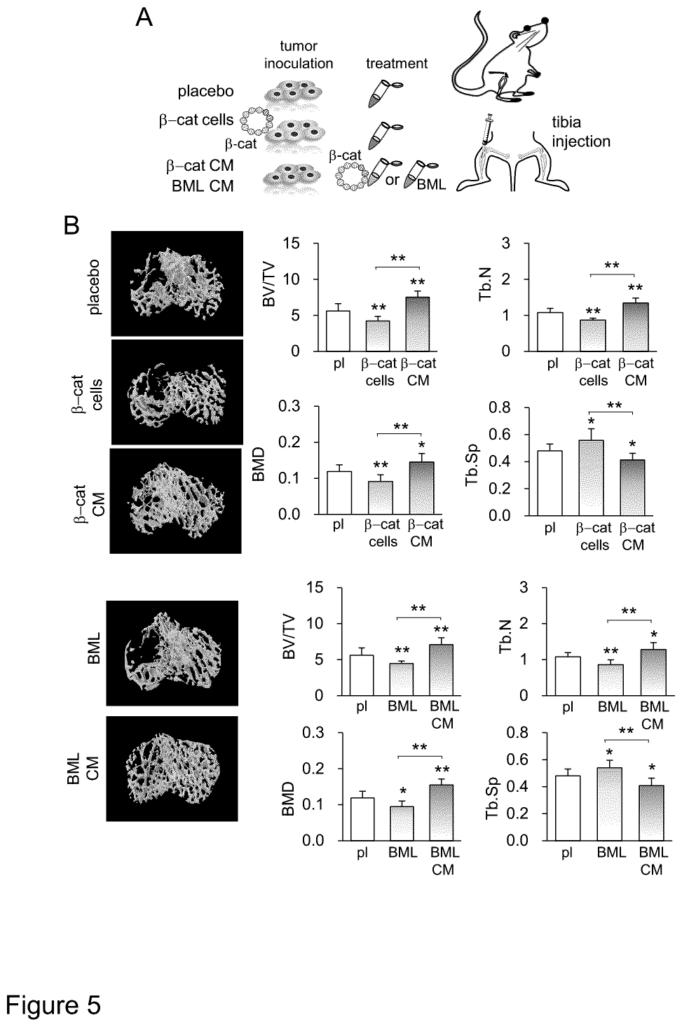

iTS CM inhibited the tumor progression and osteolysis. The effect on the tumor-invaded bone was next examined. μCT imaging of the tumor-inoculated tibia revealed that β-catenin-overexpressing iTS CM as well as BML284-treated iTS CM significantly reduced tumor-driven osteolysis (FIG. 5). In response to the iTS CM, the bone volume ratio (BV/TV), bone mineral density (BMD), and trabecular number (Tb.N) were elevated in the proximal tibia, while the trabecular separation (Tb. Sp) was reduced. These changes suggest the ability of iTS CM to protect against cancer-induced osteolysis. In contrast, the inoculation of β-catenin-overexpressing EO771 cells and BML284-treated EO771 cells stimulated bone loss by reducing BV/TV, BMD, Tb.N with an increase in Tb. Sp.

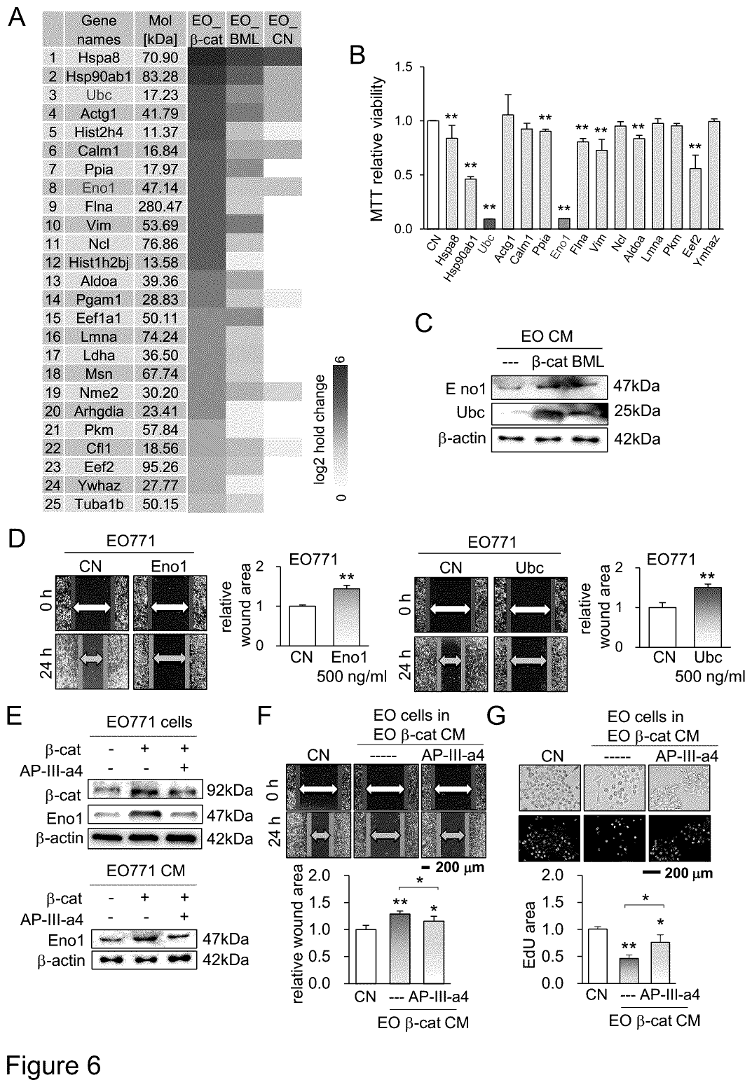

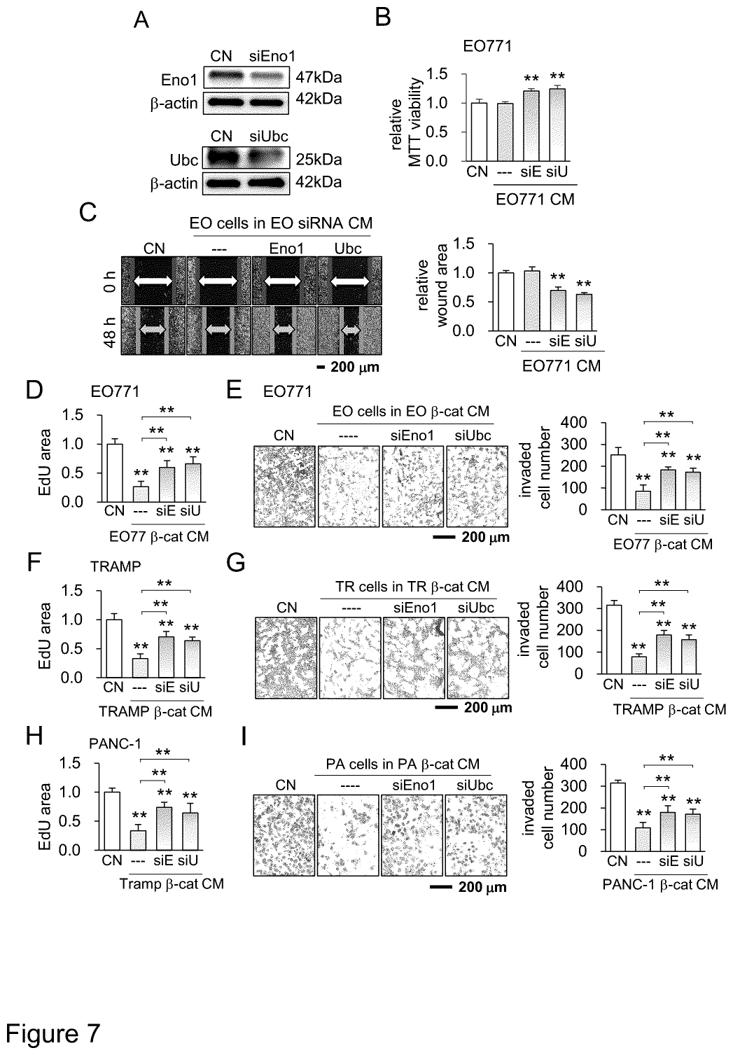

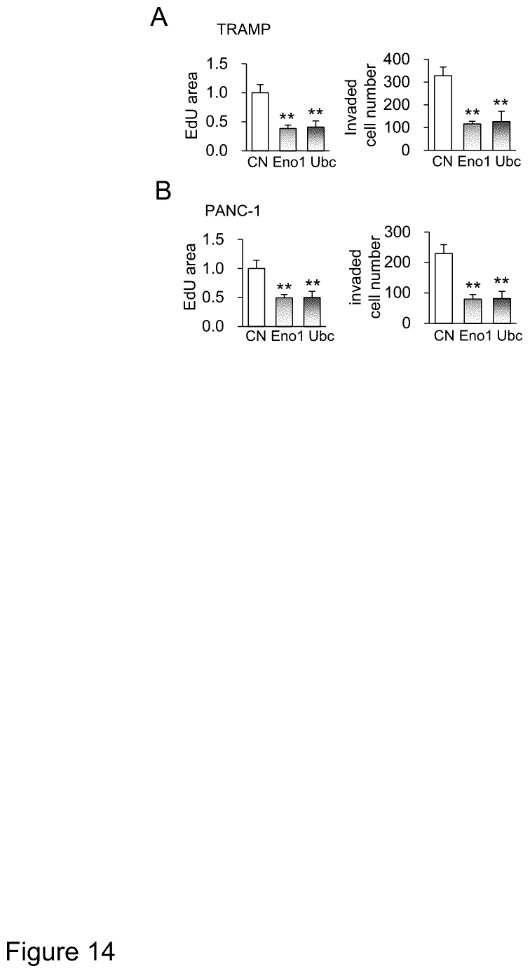

Enolase 1 and ubiquitin C were identified as tumor-suppressing factors. Mass spectrometry-based proteomics analysis identified 885 proteins in total in 4 CMs (medium control without EO771 cells, EO771 CM control, β-catenin-overexpressing CM, and BML284-treated CM), in which 97 proteins were present in β-catenin-overexpressing EO771 CM. Eighty-nine proteins were expressed higher in β-catenin-overexpressing CM than the control CM, and 25 top candidates as potential tumor suppressors are listed (FIG. 6A). Based on the availability of recombinant proteins, the effects of 15 proteins on the viability of EO771 tumor cells were evaluated (FIG. 6B). Among them, the administration of enolase 1 and ubiquitin C induced a significant decrease in the MMT-based viability. Hereafter, the main focus was on the role of these two proteins. In response to the transfection of β-catenin plasmids and BML284, EO771 mammary tumor-derived iTS CM elevated the levels of enolase 1 and ubiquitin C (FIG. 6C). They inhibited the scratch-based migration of EO771 breast cancer (FIG. 6D), and the proliferation and invasion of TRAMP prostate and PANC-1 pancreatic cancer cells (FIG. 14A&B). A pharmacological agent (ENOBLOCK™— AP-III-a4) was employed to evaluate the role of enolase 1. Its application suppressed the inhibitory effect of β-catenin-overexpressing iTS CM on the migration and invasion of mammary tumor cells (FIG. 6E-G). Also, an inhibitor of ubiquitin E3 ligase, Pomalidomide, was employed to evaluate the role of ubiquitin C. Its application interfered with the inhibitory effect of β-catenin-overexpressing iTS CM on the proliferation and invasion of mammary tumor cells (FIGS. 14C-F). Furthermore, siRNA-mediated silencing of enolase 1 and ubiquitin C promoted the proliferation and migration of EO771 mammary tumor cells (FIGS. 7A-C) and blocked the inhibitory effects of β-catenin-overexpressing CM on mammary, prostate, and pancreatic tumor cells (FIGS. 7D-I).

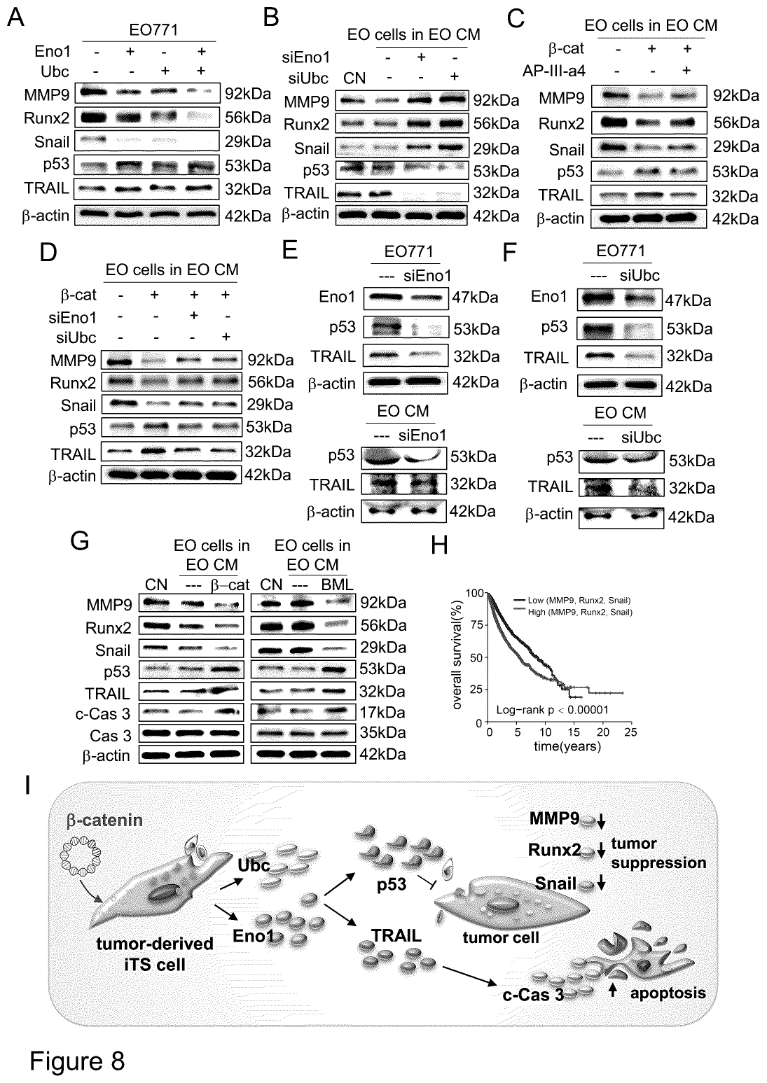

Enolase 1 and ubiquitin C downregulated tumor-promoting genes and upregulated tumor-suppressing genes. To explore the regulatory mechanism of the anti-tumor action of iTS CM, the expression of tumor-promoting proteins such as MMP9, Runx2, and Snail, a tumor-suppressing protein, p53, and an apoptosis-inducing factor, TRAIL, was examined. Western blot analysis revealed that enolase 1, ubiquitin C, and their combined application reduced MMP9, Runx2, and Snail, but elevated pro-apoptotic p53 and TRAIL (FIG. 8A). However, the inhibition of enolase 1 reversed the responses (FIG. 8B). Furthermore, their inhibitors impaired the effect of β-catenin-overexpressing iTS CM on the expression of MMP9, Runx2, Snail, p53, and TRAIL (FIG. 8C). Similarly, their siRNA-mediated knockdown in tumor cells repressed the effect of β-catenin-overexpressing iTS-CM (FIG. 8D). Also, their silencing resulted in a decrease in p53 and TRAIL in both EO771 cells and E0771-derived CM (FIGS. 8E&F).

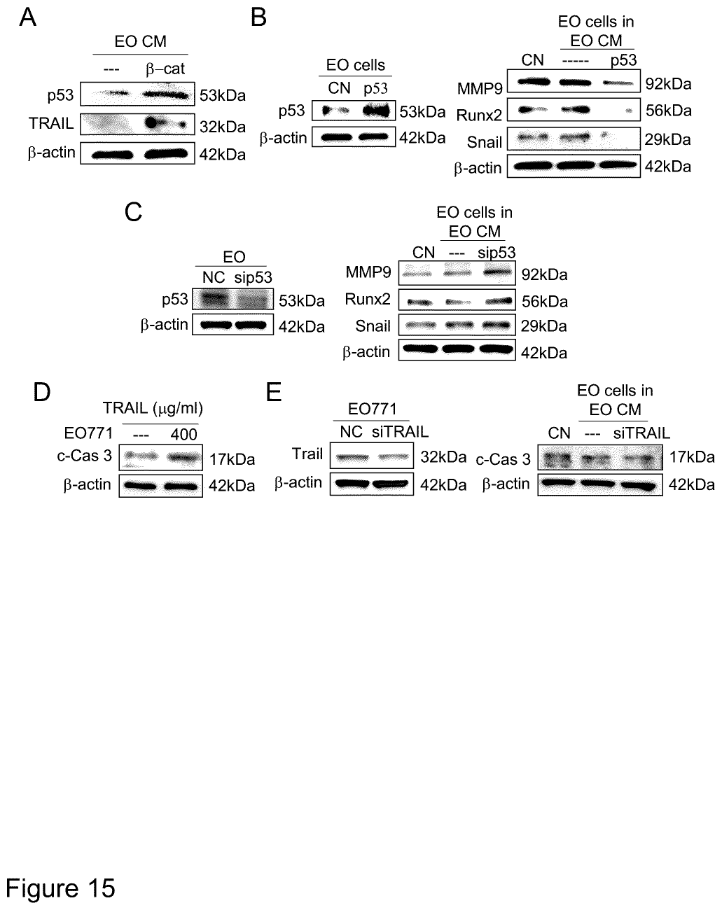

iTS CM elevated p53 and TRAIL for suppressing tumorigenic genes. In response to the transfection of β-catenin plasmids, EO771 mammary tumor-derived iTS CM increased the levels of p53 and TRAIL (FIG. 15A). It was observed that p53-overexpressing iTS CM, derived from EO771 tumor cells, markedly inhibited the expression of MMP9, Runx2, and Snail in EO771 cells (FIG. 15B), and RNA interference of p53 reversed the responses (Suppl. FIG. 7C). Furthermore, TRAIL increased the level of cleaved caspase 3 (FIG. 15D), and its RNA interference suppressed the elevation (FIG. 15E).

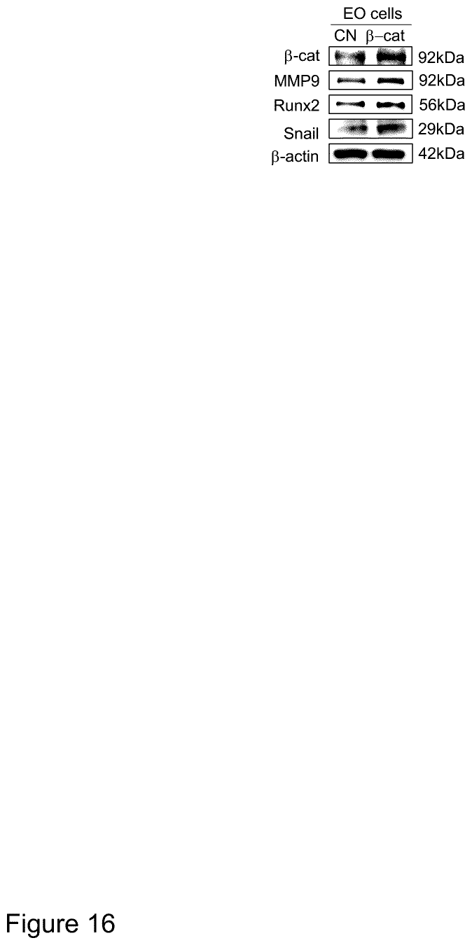

In response to EO771-derived iTS-CM by β-catenin overexpression and BML284 pre-treatment, EO771 cells downregulated MMP9, Runx2, and Snail and elevated p53, TRAIL, and cleaved caspase 3 (FIG. 8G). By contrast, the overexpression of β-catenin in EO771 cells increased the levels of MMP9, Runx2, and Snail (FIG. 16). In a pan-cancer survival analysis (9,880 primary tissue samples from 32 types of cancers), the high expression group (N=2,007), which had a higher transcript level of MMP9, Runx2, or Snail than the median value, presented a significantly unfavorable survival outcome compared to the low expression counterpart (N=2,052) (FIG. 8H).

### Example 2

Cell culture. EO771 mouse mammary tumor cells (CH3 BioSystems, Amherst, NY, USA) (11), 4T1.2 mouse mammary tumor cells (obtained from Dr. R. Anderson at Peter MacCallum Cancer Institute, Melbourne, Australia) were cultured in DMEM. MDA-MB-231 breast cancer cells, RAW264.7 pre-osteoclast cells (ATCC, Manassas, VA, USA), and MLO-A5 osteocyte-like cells (C57BL/6 background; obtained from Dr. L. Bonewald at Indiana University, IN, USA) were grown in Minimum Essential Medium Eagle—α Modification (“αMEM”). Murine MSCs derived from the bone marrow of the C57BL/6 strain (Envigo RMS, Inc., Indianapolis, IN, USA) were cultured in MESENCULT™ culture medium (Stem Cell Technology, Cambridge, MA, USA). Human adipose mesenchymal stem cells (SCC038, Sigma-Aldrich, Missouri, USA) were cultured in αMEM. The culture media was supplemented with 10% FBS and antibiotics (100 units/ml penicillin, and 100 μg/ml streptomycin; Life Technologies, Grand Island, NY, USA), and cells were maintained at 37° C. and 5% CO₂. Conditioned medium (CM) was prepared with antibiotics and a fraction of FBS with 3 kDa or smaller. After one day of incubation, the medium was condensed 10-fold using a filter with a cut-off molecular weight at 3 kDa (Thermo-Fisher, Waltham, MA, USA).

EdU assay. Using a procedure previously reported, approximately 1,000 cells were seeded in 96-well plates on day 1. CM was added on day 2 and cellular proliferation was examined with a fluorescence-based cell proliferation kit (CLICK-IT™ EdU ALEXA FLUOR™ 488 Imaging Kit; Thermo-Fisher, Waltham, MA, USA) on day 4. After fluorescent labeling, the number of fluorescently labeled cells was counted and the ratio to the total number of cells was determined.

TRANSWELL® invasion assay. In a TRANSWELL® invasion assay, approximately 5×10⁴ cells in 200 μL serum-free DMEM were placed on the upper TRANSWELL® chamber (Thermo Fisher Scientific, Waltham, MA, USA) with MATRIGEL™ (100 μg/ml), and 800 μL of CM was added in the lower chamber. After 48 h, the cells that had invaded the lower side of the membrane were stained with Crystal Violet. At least five randomly chosen images were taken, and the average number of stained cells was determined.

Scratch assay. A wound-healing scratch assay was conducted to evaluate 2-dimensional migratory behavior. Approximately 4×10⁵ cells were seeded in 12-well plates. After cell attachment, a plastic pipette tip was used to scratch a gap on the cell layer. After the removal of the floating cells, CM was added. Images of the cell-free scratch zone were collected at 0 h, and the areas newly occupied with cells were determined 24-48 h after scratching. The areas were quantified with IMAGE J™ (National Institutes of Health, Bethesda, MD, USA).

Osteoclast differentiation assay. The differentiation assay of RAW264.7 pre-osteoclasts was performed in a 12-well plate. During the 6-day incubation of pre-osteoclast cells in 40 ng/ml of RANKL, the culture medium was exchanged once on day 4. Adherent cells were fixed and stained with a tartrate-resistant acid phosphate (TRAP)-staining kit (Sigma-Aldrich, Missouri, USA), according to the manufacturer's instructions. TRAP-positive multinucleated cells (>3 nuclei) were identified as mature osteoclasts and counted.

Western blot analysis. Cultured cells were lysed in a radio-immunoprecipitation assay (RIPA) buffer and proteins were fractionated by 10-15% SDS gels and electro-transferred to polyvinylidene difluoride (PVDF) transfer membranes (Millipore, Billerica, MA, USA). The membrane was incubated overnight with primary antibodies and then with secondary antibodies conjugated with horseradish peroxidase (Cell Signaling, Danvers, MA, USA). Antibodies against c-Myc, Oct4, Sox2, Klf4, Lrp5, Runx2, Snail, TGFβ, Eno1, Eef2, vinculin (Cell Signaling, Danvers, MA, USA), MMP9, NFATc1, cathepsin K (Santa Cruz, Dallas, TX, USA), TRAIL (Novus, Centennial, CO, USA), p53 (Invitrogen, Carlsbad, CA, USA), Hsp90ab1 (Abcam, Cambridge, UK), Kdm3a (Thermo-Fisher Scientific, Waltham, MA, USA) and β-actin as a control (Sigma, St. Louis, MO, USA) were employed. The level of proteins was determined using a SUPERSIGNAL™ west femto maximum sensitivity substrate (Thermo-Fisher Scientific, Waltham, MA, USA), and a luminescent image analyzer (LAS-3000, Fuji Film, Tokyo, Japan) was used to quantify signal intensities.

Plasmid transfection and RNA interference. The overexpression of c-Myc, Oct4, Sox2, and Klf4 was conducted by transfecting plasmids (#17758, #19778, #26817, #26815; Addgene, Cambridge, MA, USA), while blank plasmids (FLAG-HA-pcDNA3.1; Addgene) were used as a control. RNA interference was conducted using siRNA specific to Oct4, Eno1, Hsp90ab1, Eef2, and vinculin (115304, s234544, s67897, 157269, 186995; Thermo-Fisher) with a negative siRNA (Silencer Select #1, Thermo-Fisher) as a nonspecific control using the procedure previously described.

3D spheroid competition assay and ex vivo tissue assay. In a three-dimensional spheroid assay, tumor spheroids were formed by culturing cells in the U-bottom low-adhesion 96-well plate (S-Bio, Hudson, NH, USA) at 1×10⁴ cells/well for 4T1.2 cells). To evaluate the effect of iTS CM, the medium was replaced by CM, and spheroid images were captured after 48 h. fluorescently labeled 4T1.2 cells were prepared by culturing them with a green (#4705, Sartorius, Gottingen, Germany) or red fluorescent dye (#4706) for 20 min at 37° C. Cells were then harvested as a pellet by centrifuging at 1000 rpm for 5 min. Cells were imaged every 24 h, and the area was calculated with IMAGE J™. In the ex vivo tissue assay, the usage of human breast cancer tissues was approved by the Indiana University Institutional Review Board, and the tissues were received from Simon Cancer Center Tissue Procurement Core. A sample (˜1 g) was manually fragmented with a scalpel into small pieces (0.5˜0.8 mm in length), which were grown in DMEM with 10% FBS and antibiotics for a day. iTS cell-derived CM was then added for two additional days and a change in the fragment size was determined.

Animal models. The experimental procedures were approved by the Indiana University Animal Care and Use Committee and were complied with the Guiding Principles in the Care and Use of Animals endorsed by the American Physiological Society. Mice were randomly housed five per cage by a stratified randomization procedure based on body weight. Mouse chow and water were provided ad libitum. BALB/c female mice (˜8 weeks, Envigo, Indianapolis, IN, USA) were divided into 3 groups (placebo, Oct4 CM, and OAC2 CM groups) in the first experiment, while they were divided into 3 groups (placebo, c-Myc CM, and Oct4/c-Myc CM groups) in the second experiment. In the mouse model of a mammary tumor, BALB/c female mice received a subcutaneous injection of 4T1.2 cells (3.0×10⁵ cells in 50 μL PBS) to the mammary fat pad on day 1. For the tibial osteolysis mouse model, BALB/c female mice per group received an intra-tibial injection of 4T1.2 cells (3.0×10⁵ cells in 20 μL PBS) to the right tibia on day 1.

For examining the efficacy of iTS-derived CM, CM was condensed by a filter with a cutoff molecular weight of 3 kDa and the 10-fold condensed CM (50 μl re-suspended in PBS) was intravenously injected from the tail vein at the same time from day 2. The animals were sacrificed on day 14 and mammary tumors and tibiae were harvested.

μCT imaging and histology. The tibiae were harvested for μCT imaging and histology. Micro-computed tomography was performed using SKYSCAN™ 1172 (Bruker-MicroCT, Kontich, Belgium). Using manufacturer-provided software, scans were performed at pixel size 8.99 μm and the images were reconstructed (nRecon v1.6.9.18) and analyzed (CTan v1.13). Using μCT images, trabecular bone parameters such as bone volume ratio (BV/TV), bone mineral density (BMD), trabecular number (Tb.N), and trabecular separation (Tb.Sp) were determined in a blinded fashion. In histology, H&E staining was conducted. Of note, normal bone cells appeared in a regular shape with round and deeply stained nuclei, while tumor cells were in a distorted shape with irregularly stained nuclei.

Mass spectrometry-based proteomics analysis. The freeze-dried pellet samples were harvested from the culture medium using 4T1.2 mouse mammary tumor cells, which were treated with Oct4 plasmid and OAC2, respectively. Proteins in CM were analyzed in the DIONEX ULTIMATE™ 3000 RSLC nano system combined with the Q-exactive high-field hybrid quadrupole orbitrap mass spectrometer (Thermo Fisher Scientific). Proteins were first digested on-beads using trypsin/LysC, and this digestion was performed in 50 mM ammonium bicarbonate buffer instead of urea. Digested peptides were then desalted using mini spin C18 spin columns (The Nest Group, Southborough, MA, USA) and separated using a trap and 50-cm analytical columns. Raw data were processed using MAXQUANT™ (v1.6.3.3) against the Uniprot mouse protein database at a 1% false discovery rate allowing up to 2 missed cleavages. MS/MS counts were used for relative protein quantitation. Proteins identified with at least 1 unique peptide and 2 MS/MS counts were considered for the final analysis.

The recombinant proteins employed for in vitro assays included Eno1, Pkm, Ppia, Hspa5, Aldoa, Lgals1, Filamin A, Eef2, Tpi1, Pgk1, Pfn1, Plec, Nme2, Eef1a1, Myh9, vcl, Hsp90aa1, Calm1 (MBS2009113, MBS8249600, MBS286137, MBS806904, MBS8248528, MBS2086775, MBS962910, MBS1213669, MBS144173, MBS717266, MBS956765, MBS2031199, MBS145412, MBS2033168, MBS717396, MBS957842, MBS142709, MB S2018713; MyBioSource, San Diego, CA, USA), actin gamma 1, Actn4, Hspa8 (H00000071-P01, H00000081-P01, NBP1-30278; Novus, Littleton, CO, USA), and Hsp90ab1 (OPCA05157; Aviva System Biology, San Diego, CA, USA).

Statistical analysis. For cell-based experiments, three or four independent experiments were conducted and data were expressed as mean±S.D. In animal experiments, the sample size was chosen to achieve a power of 80% with p<0.05. The primary experimental outcome was tumor weight for the mammary fat pad experiment and the bone volume ratio (BV/TV) for the tibia experiment. The secondary experimental outcome was tumor size for the mammary fat pad experiment and the trabecular number (Tb.N) for the tibia experiment. Statistical significance was evaluated using a one-way analysis of variance (ANOVA). Post hoc statistical comparisons with control groups were performed using Bonferroni correction with statistical significance at p<0.05. The single and double asterisks in the figures indicate p<0.05 and p<0.01, respectively.

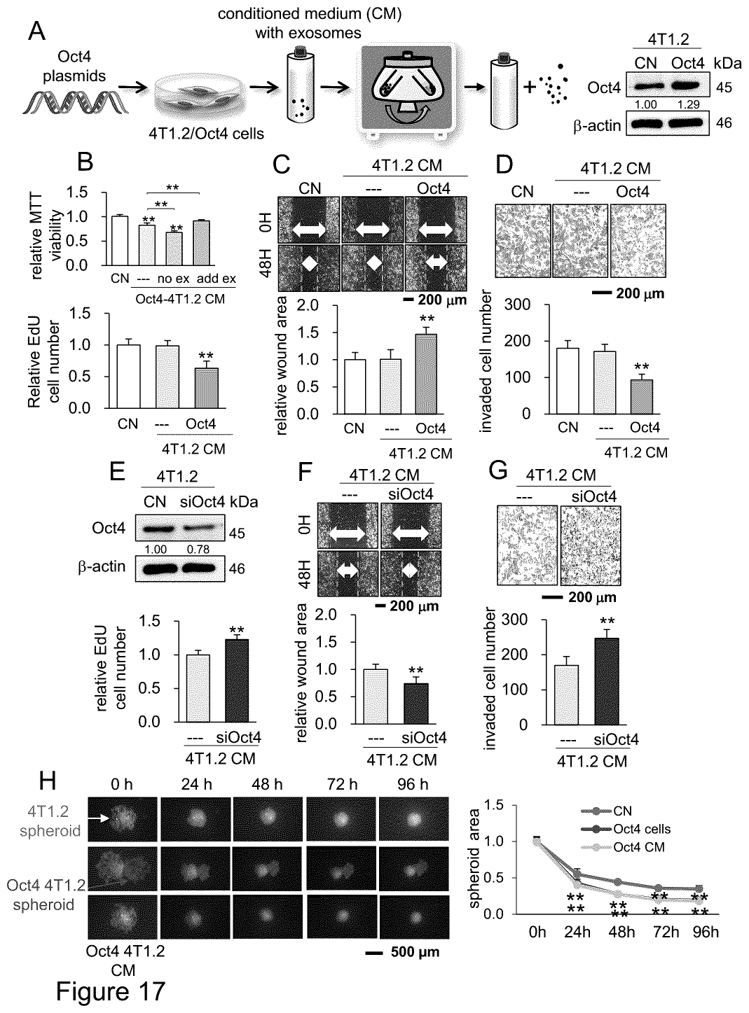

Tumor suppression in vitro by Oct4-overexpressing tumor cell-derived CM. To test the tumor-suppressing effect of Oct4, CM from Oct4-overexpressing 4T1.2 mammary tumor cells was harvested and ultra-centrifuged to remove exosomes. It was observed that Oct4-overexpressing CM (Oct4 CM) reduced the MTT-based viability of 4T1.2 parental cells and the removal of exosomes enhanced the reduction (FIG. 17A&B). Oct4 CM also reduced the EdU-based proliferation, scratch-based migration, and TRANSWELL® invasion of 4T1.2 parental cells (FIG. 17B-D). In contrast, Oct4-silenced CM reversed the responses and acted as a tumor promoter (FIG. 17E-G). In the three-dimensional tumor spheroid assay, 4T1.2 tumor spheroids were shrunk by Oct4-overexpressing tumor spheroids and their CM (FIG. 17H). The CM was employed after the ultracentrifugation.

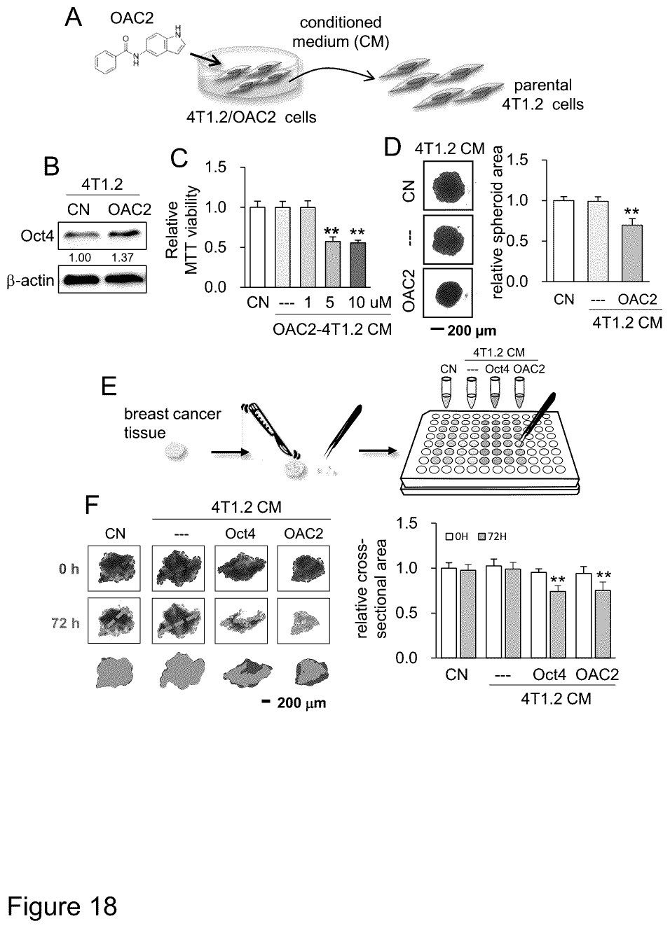

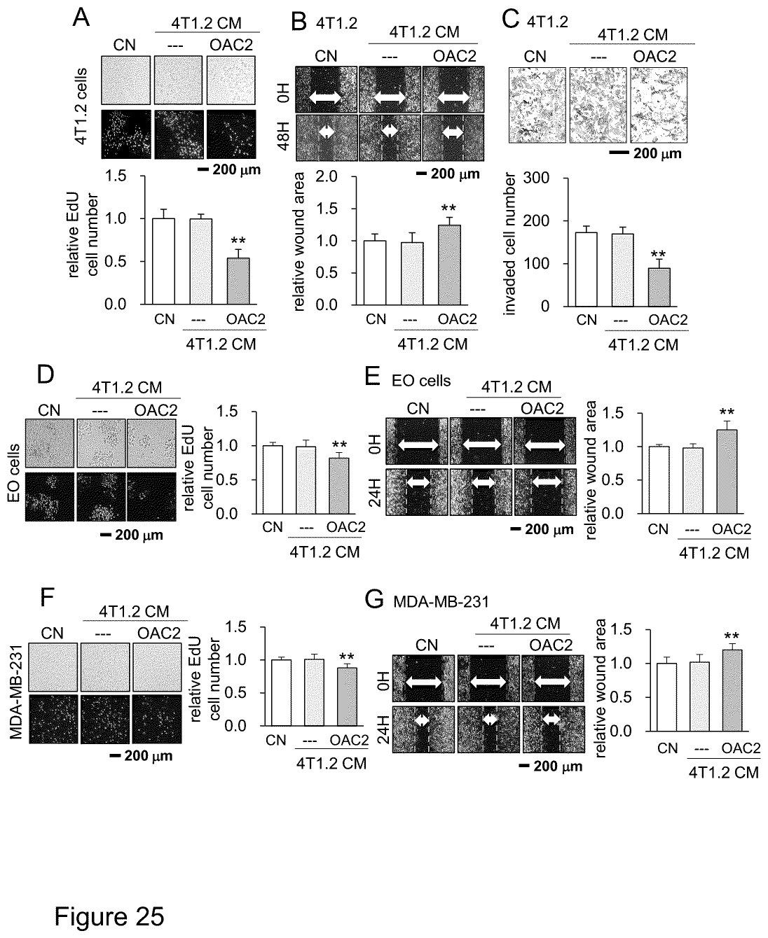

Tumor suppression in vitro by OAC2-treated tumor cell-derived CM. Next, OAC2, a pharmacological agent for activating Oct4, was employed. OAC2-treated 4T1.2-derived CM (OAC2 CM) also reduced the MTT-based viability of 4T1.2 parental cells and the growth of 4T1.2 tumor spheroids (FIG. 18A-D). Furthermore, in the ex vivo breast cancer tissue assay, the size of cancer tissue fragments, freshly isolated from a patient with breast cancer, was significantly reduced by the application Oct4 CM and OAC2-treated CM (FIG. 18E&F). The tumor-suppressing action of 4T1.2-derived OAC2 CM was observed not only for 4T1.2 parental cells but also for EO771 mammary tumor cells and MDA-MB-231 breast cancer cells (FIG. 25).

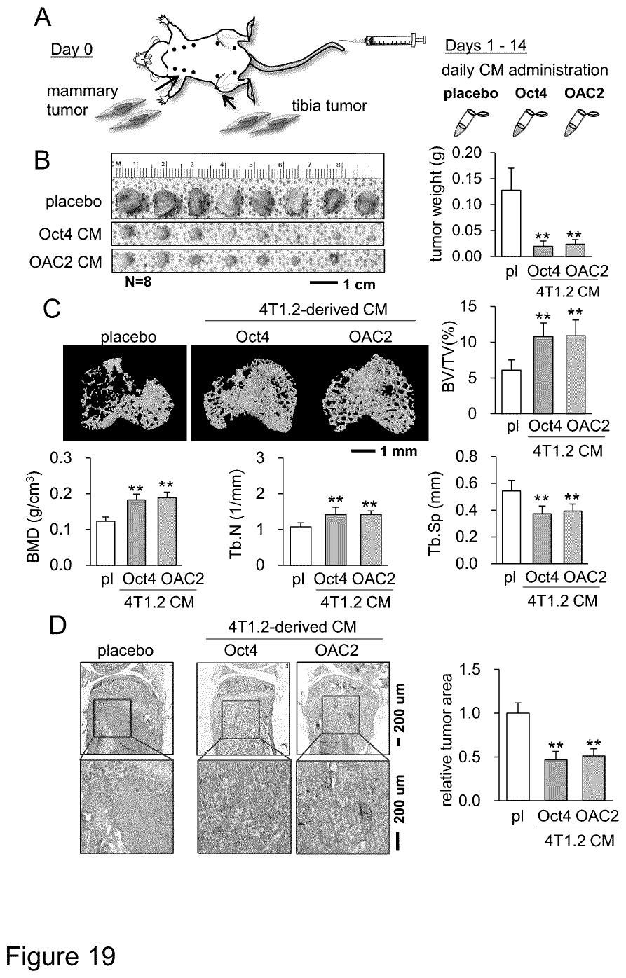

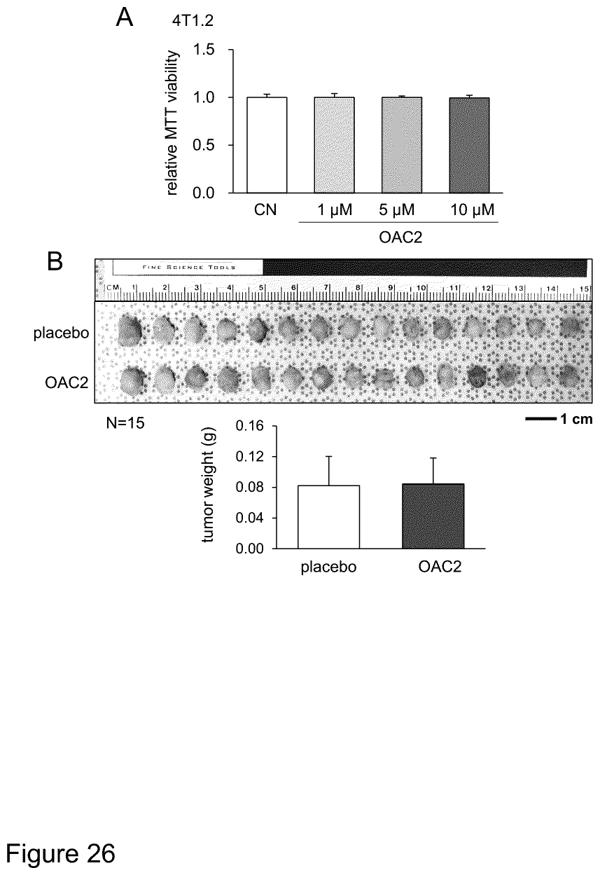

Tumor suppression and bone protection in vivo by Oct4 CM and OAC2 CM. Using the mouse models of mammary tumors and tibial osteolysis, the effects of Oct4 and OAC2 were examined in vivo, in which Oct4 CM or OAC2 CM was administered daily as an intravenous injection (FIG. 19A). Notably, both Oct4 CM and OAC2 CM significantly reduced the growth of mammary tumors (FIG. 19B). Furthermore, these CMs prevented bone loss by elevating bone volume ratio, bone mineral density, and trabecular numbers, while decreasing trabecular separation that represented the spacing in trabecular bone (FIG. 19C). Histological analysis with H&E-stained bone sections also supported the anti-tumor effect of CMs and the tumor-invaded areas were markedly reduced by Oct4 CM and OAC2 CM (FIG. 19D). In contrast, the direct application of OAC2 to 4T1.2 cells or the daily injection of OAC2 to the mouse model of mammary tumors did not alter the proliferation of tumor cells or the progression of tumors (FIG. 26).

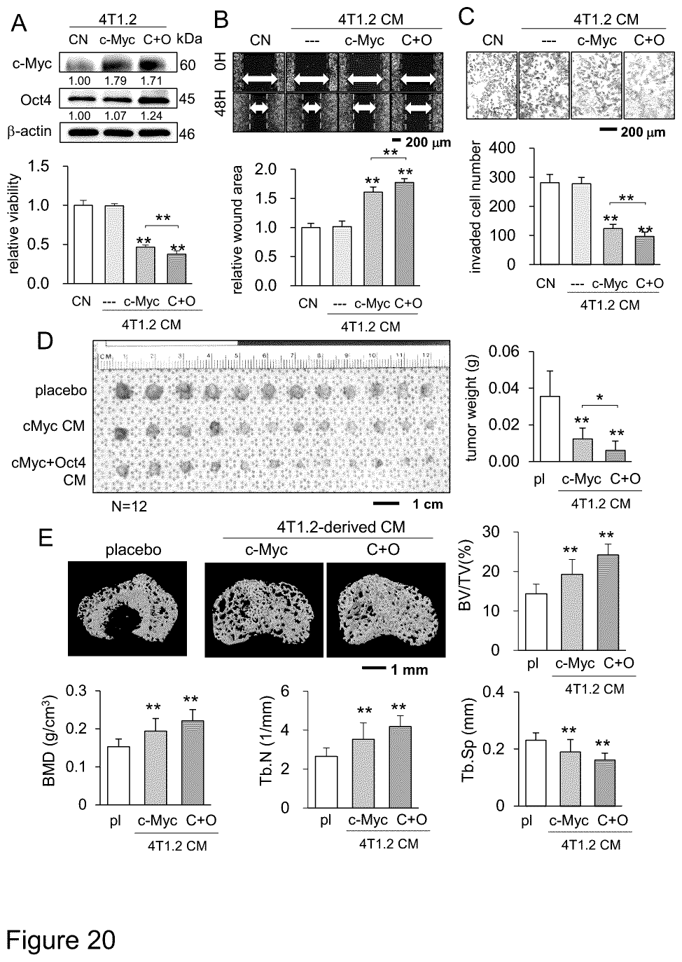

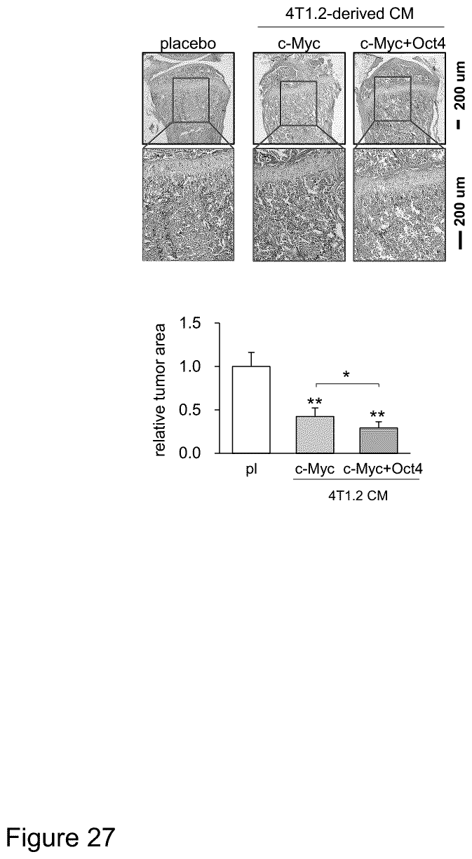

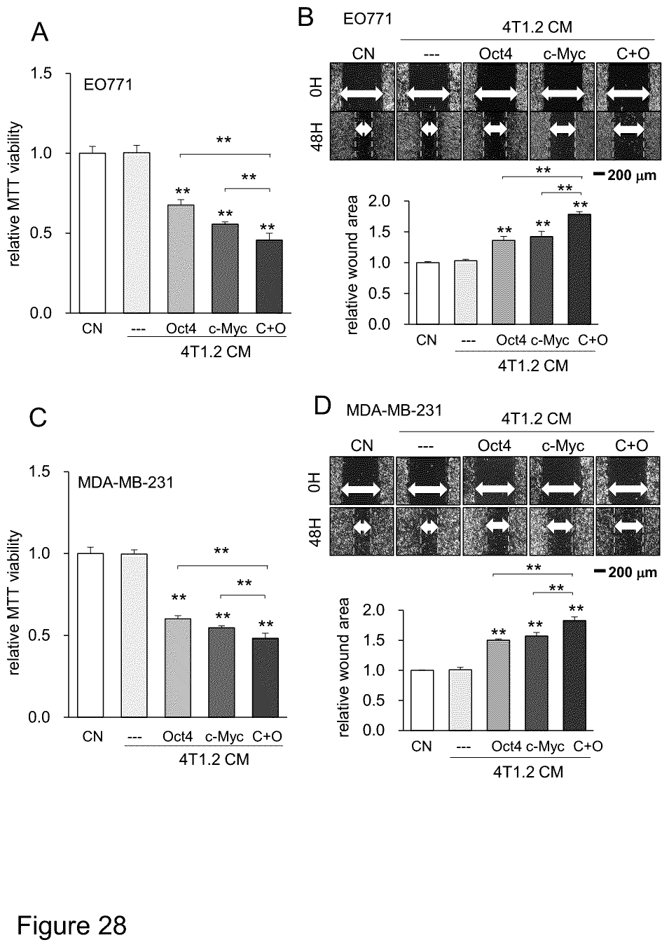

Tumor suppression by c-Myc and Oct4 CMs. Encouraged by the result with Oct4 CM, the effect of c-Myc-overexpressing 4T1.2 tumor cell-derived CM (c-Myc CM) was examined. The result was affirmative and c-Myc CM also reduced MTT-based viability, scratch-based migration, and TRANSWELL® invasion of parent tumor cells, and the simultaneous overexpression of c-Myc and Oct4 strengthened the inhibitory action (FIG. 20A-C). Consistently, in vivo data with the mouse models of mammary tumors and bone osteolysis supported the induced tumor-suppressing capability, and c-Myc CM significantly inhibited the growth of mammary tumors and the destruction of trabecular bone in the tibia (FIG. 20D&E; FIG. 27). Moreover, the double overexpression of Oct4 and c-Myc (Oct4/c-Myc CM) enhanced inhibitory actions. The anti-tumor actions of Oct4 CM, c-Myc CM, and Oct4/c-Myc CM were also detected in EO771 mammary cells as well as MDA-MB-231 breast cancer cells (FIG. 28).

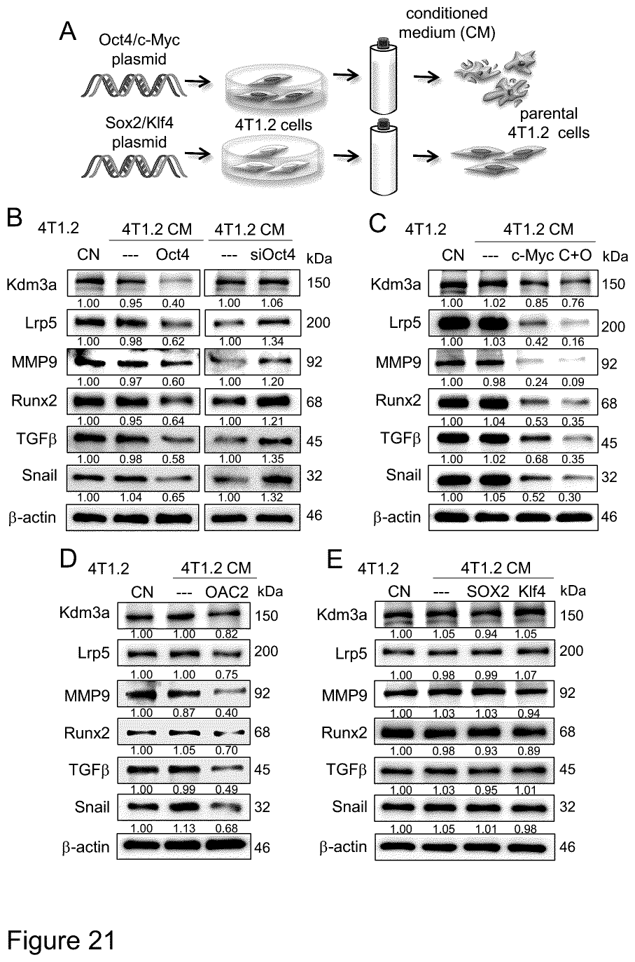

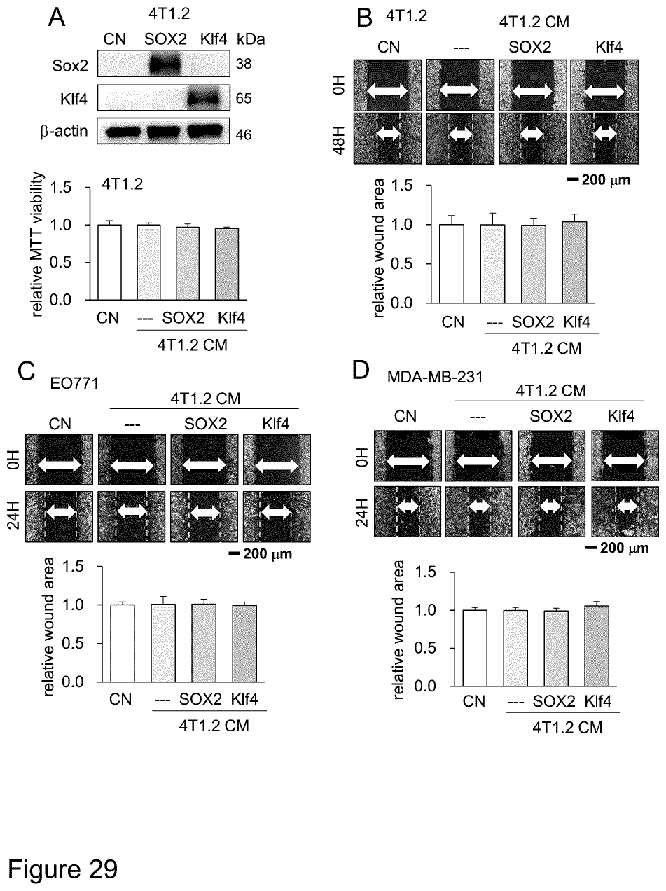

Undetectable tumor-suppressing effects of Sox2 and Klf4. While Oct4 and c-Myc converted tumor cells into iTS cells, the overexpression of Sox2 or Klf4 did not show the anti-tumor capability. Sox2 CM and Klf4 CM did not induce detectable changes in MMT-based viability and scratch-based migration in 4T1.2, E0771, and MDA-MB-231 cell lines (FIG. 29). Mechanistically, the overexpression of Oct4 and c-Myc in tumor cells altered the expression of downstream oncogenic genes in parental tumor cells but Sox2 and Klf4 did not. 6 oncogenic genes (Kdm3a, Lrp5, MMP9, Runx2, TGFβ, and Snail) were selected, and their expression levels in response to CMs were evaluated. Of note, Kdm3a is a histone demethylase to regulate the availability of chromatin, while Lrp5 is a co-receptor of Wnt signaling and MMP9 is a matrix metalloproteinase to promote tumor migration. Runx2 and TGFβ assist tumor progression, and Snail is involved in EMT. The result revealed that Oct4 CM and c-Myc CM downregulated all of these genes in 4T1.2 parental cells. Oct4/c-Myc CM (double transfection) further downregulated them, while Oct4 siRNA-treated CM suppressed the downregulation (FIG. 21A-C). Also, OAC2 CM presented the same inhibitory action (FIG. 21D). Most importantly, however, no detectable change was observed in the expression of the selected genes by Sox2 CM and Klf4 CM (FIG. 21E).

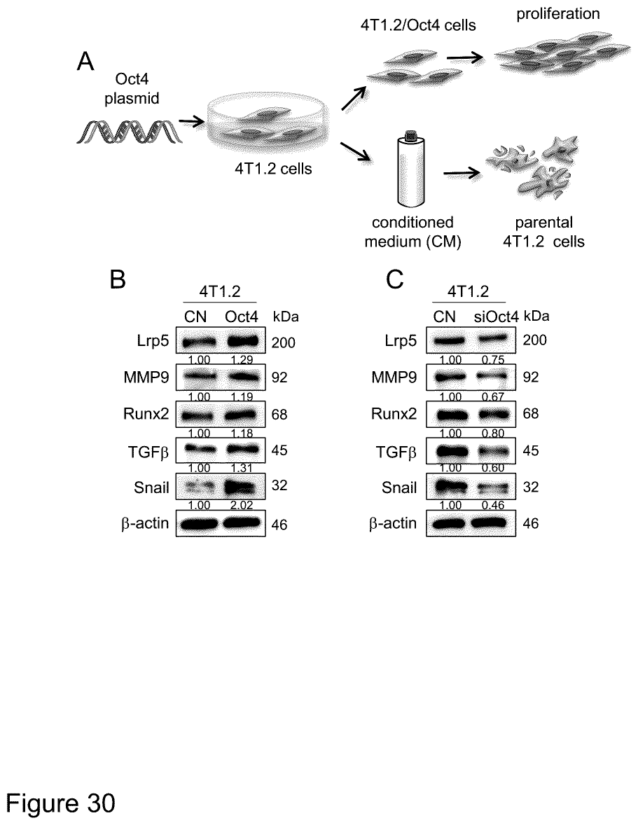

Overexpression of Oct4 induced two opposite effects in tumor cells and Oct4 CM-treated tumor cells. It was shown that the selected tumorigenic genes (Lrp5, MMP9, Runx2, TGFβ, and Snail) were downregulated in Oct4 CM-treated 4T1.2 parental cells. In contrast, the overexpression of Oct4 in 4T1.2 cells elevated these tumorigenic genes in Oct4-overexpressing 4T1.2 cells and the silencing of Oct4 in 4T1.2 cells reduced them in Oct4-silenced tumor cells (FIG. 30). Collectively, the role of the inducer of iTS cells, Oct4, is stimulatory to iTS cells and inhibitory to neighboring tumor cells, which are exposed to their CM.

Enolase 1, Hsp90ab1, Eef2, and vinculin as tumor suppressor candidates in CM. To determine the critical proteins for the tumor-suppressing action of Oct4 CM and OAC2 CM, mass spectrometry-based proteomics analysis was conducted. In 4 CMs (medium control, CM control, Oct4 CM, and OAC2 CM), 395 proteins were identified by mass spectrometry and 100 proteins were detected at a higher level in Oct4 CM and OAC2 CM than the control CM. Table 1 is a list of 100 proteins that were expressed higher in Oct4-overexpressing OAC2-treated CMs than the control CM in mass spectrometry-based proteonomics analysis.

TABLE 1

| | | Expression Level | | |
|---|---|---|---|---|
| | Mol. Wt. | (MS/MS Count) | | |
| Gene Name | (kDa) | CN | Oct4 | OAC2 |
| Hspa8 | 70.9 | 74 | 328 | 250 |
| Eno1 | 47.1 | 61 | 296 | 236 |
| Pkm | 57.8 | 51 | 192 | 166 |
| Ppia | 18.0 | 44 | 180 | 128 |
| Hspa5 | 72.4 | 46 | 122 | 158 |
| Hsp90ab1 | 83.3 | 37 | 121 | 120 |
| Aldoa | 39.4 | 36 | 123 | 105 |
| Lgals1 | 14.9 | 19 | 99 | 87 |
| Flna | 280.5 | 31 | 102 | 91 |
| Eef2 | 95.3 | 13 | 80 | 62 |
| Tpi1 | 32.2 | 12 | 72 | 55 |
| Actn4 | 105.0 | 9 | 63 | 54 |
| Vcp | 89.3 | 9 | 66 | 47 |
| Actg1 | 41.8 | 32 | 70 | 88 |
| Pgk1 | 44.6 | 8 | 59 | 48 |
| Flnb | 277.8 | 5 | 61 | 38 |
| Pfn1 | 14.9 | 9 | 68 | 39 |
| Plec | 506.5 | 13 | 63 | 46 |
| Ldha | 36.5 | 13 | 63 | 46 |
| Nme2 | 30.2 | 17 | 67 | 43 |
| Eef1a1 | 50.1 | 29 | 73 | 59 |
| Myh9 | 226.4 | 13 | 36 | 52 |
| Vcl | 116.7 | 10 | 43 | 39 |
| Hsp90aa1 | 84.8 | 9 | 42 | 36 |
| Calm1 | 16.8 | 18 | 51 | 44 |
| Cfl1 | 18.6 | 13 | 47 | 38 |
| GAPDH; Gapdh; m3839 | 35.8 | 17 | 52 | 40 |
| Lmna | 74.2 | 30 | 67 | 50 |
| Pdia3 | 56.7 | 15 | 47 | 39 |
| Gpi; Gpi1 | 62.8 | 2 | 32 | 25 |
| Msn | 67.7 | 19 | 50 | 41 |
| Pgam1 | 28.8 | 6 | 36 | 28 |
| Arhgdia | 23.4 | 11 | 38 | 35 |
| Fasn | 272.4 | 3 | 27 | 29 |
| Tln1 | 272.1 | 0 | 27 | 23 |
| Tuba1b; Tuba1c; Tuba1a | 50.2 | 16 | 35 | 46 |
| Tkt | 67.6 | 16 | 44 | 37 |
| Ywhae | 29.2 | 11 | 39 | 31 |
| Tagln2 | 22.4 | 7 | 37 | 24 |
| Dpysl2 | 62.3 | 8 | 34 | 27 |
| Prdx6 | 24.9 | 9 | 40 | 23 |
| Flnc | 291.1 | 0 | 25 | 16 |
| Gm1821; Ubc; Uba52; Ubb | 17.2 | 23 | 48 | 38 |
| Eif5a; Eif5a2 | 16.3 | 6 | 35 | 17 |
| Actn1 | 103.1 | 3 | 24 | 21 |
| Hspa4 | 94.2 | 11 | 35 | 26 |
| Ywhaz | 27.8 | 12 | 34 | 28 |
| Pabpc1 | 70.7 | 2 | 27 | 15 |
| Stip1 | 62.5 | 10 | 37 | 21 |
| Tubb5 | 49.7 | 12 | 22 | 37 |
| Tpm3; Tpm3-rs7 | 29.0 | 20 | 38 | 37 |
| Iqgap1 | 188.8 | 0 | 16 | 18 |
| Prdx1 | 22.2 | 7 | 26 | 22 |
| Fkbp4 | 51.6 | 3 | 24 | 15 |
| Lgals3 | 19.9 | 14 | 39 | 22 |
| Mdh2 | 35.6 | 0 | 13 | 19 |
| Sptan1 | 282.9 | 0 | 14 | 18 |
| Hnrnpa2b1 | 37.4 | 14 | 31 | 29 |
| Cmpk2 | 50.0 | 0 | 26 | 5 |
| Ak2 | 26.5 | 4 | 22 | 16 |
| Cct8; Cctq | 59.6 | 0 | 20 | 10 |
| Idh1 | 46.7 | 2 | 15 | 18 |
| Cct3 | 60.6 | 2 | 18 | 15 |
| Uba1 | 117.8 | 0 | 16 | 13 |
| Gdi2 | 50.5 | 0 | 17 | 12 |
| Rplp2 | 11.7 | 2 | 23 | 10 |
| Hist1hsbj; Hist1h2bk | 13.6 | 11 | 25 | 25 |
| Psma7; Psma8 | 27.9 | 2 | 16 | 16 |
| Mtap | 31.1 | 4 | 20 | 15 |
| Wdr1 | 66.4 | 2 | 19 | 12 |
| G3bp1 | 56.2 | 0 | 17 | 10 |
| Hist1h2ah; Hist1h2aa | 13.7 | 8 | 20 | 22 |
| Gm | 63.5 | 0 | 12 | 14 |
| Srsf1 | 28.3 | 0 | 13 | 13 |
| Pgd | 53.3 | 2 | 18 | 12 |
| Psme1 | 27.4 | 0 | 17 | 9 |
| Lap3 | 56.1 | 0 | 18 | 8 |
| Atic | 64.2 | 3 | 14 | 17 |
| Eef1g | 50.1 | 6 | 22 | 15 |
| Ahcy | 47.7 | 0 | 16 | 9 |
| Cltc; mKlAA0034 | 192.0 | 0 | 11 | 13 |
| Anxa2 | 38.6 | 6 | 18 | 18 |
| Serpinb6a; Serpinb6 | 42.6 | 0 | 13 | 11 |
| Pnp; Pnp2 | 32.3 | 0 | 21 | 3 |
| Psat1 | 40.5 | 2 | 15 | 12 |
| Pls3 | 70.6 | 0 | 13 | 10 |
| Ctsl | 37.6 | 42 | 44 | 62 |
| Ncl | 76.9 | 15 | 24 | 28 |
| Ctsb | 37.3 | 3 | 16 | 12 |
| Pa2g4 | 43.7 | 2 | 15 | 11 |
| Ckb | 42.7 | 0 | 13 | 9 |
| Got1 | 46.2 | 0 | 14 | 8 |
| Cct5 | 59.6 | 0 | 14 | 8 |
| Sptbn1 | 274.2 | 0 | 10 | 11 |
| Mif | 12.5 | 7 | 18 | 17 |
| Gstp1; Gstp2 | 23.6 | 2 | 14 | 11 |
| Eprs | 170.0 | 0 | 12 | 9 |
| Hmga1 | 11.6 | 2 | 15 | 10 |
| Cct2 | 57.5 | 0 | 14 | 7 |
| Vars | 140.2 | 2 | 17 | 8 |

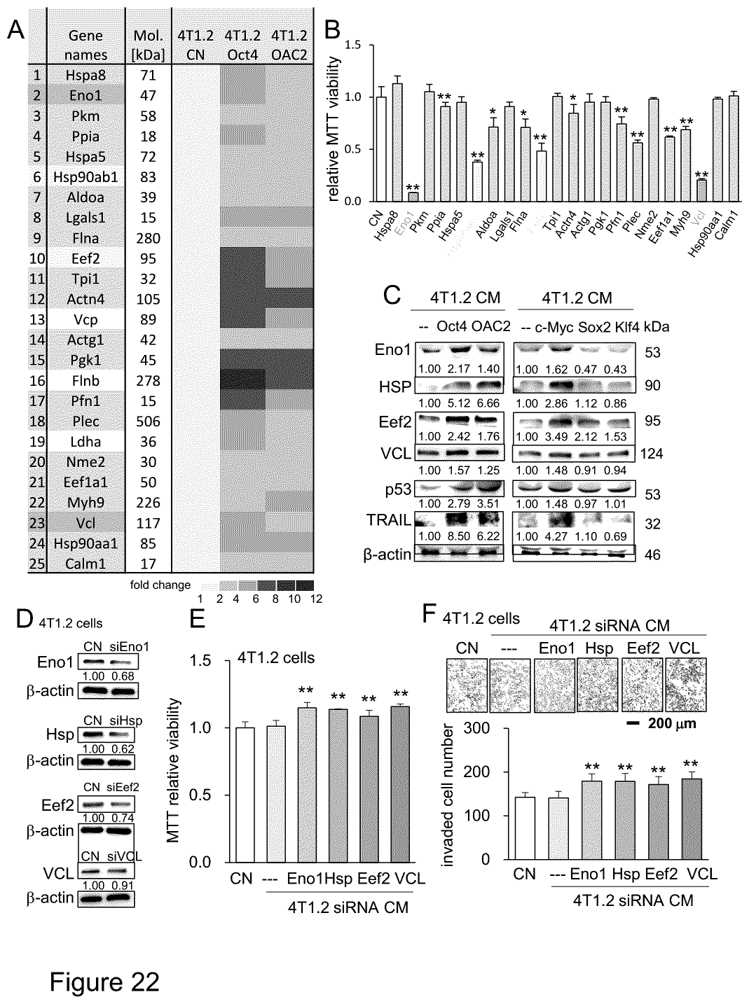

As potential tumor suppressors, 25 candidates were examined, and based on the availability of recombinant proteins, the effects of 22 proteins were evaluated using the MTT assay (FIG. 22A). Among 22 proteins, 12 proteins induced a statistically significant decrease in viability of parental 4T1.2 cells (FIG. 22B). The most striking reduction (50% or more) was detected with four proteins (enolase 1, Hsp90ab1, Eef2, and vinculin). The expression levels of these putative tumor suppressors, together with p53 and Trail, were elevated in Oct4 CM, OAC2 CM, and c-Myc CM, but not in Sox2 CM and Klf4 C (FIG. 22C). Consistently, the administration of CM, harvested from 4T1.2 cells that were treated with siRNAs specific to enolase 1, Hsp90ab1, Eef2, and vinculin, elevated the MTT-based viability and TRANSWELL® invasion of 4T1.2 cells (FIG. 22D-F). Hereafter, the tumor-suppressing role of these four proteins is examined.

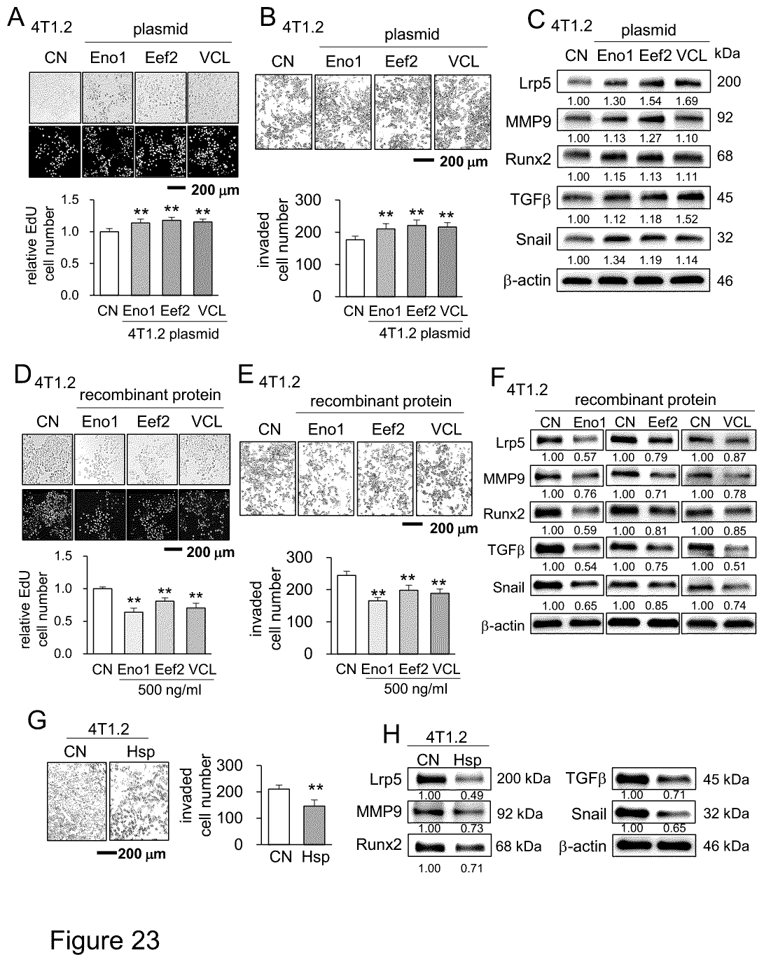

Differential roles of intracellular and extracellular Enolase 1, Hsp90ab1, Eef2, and vinculin. In analyzing the role of the predicted tumor suppressors, their roles intracellularly and extracellularly were examined and dichotomous roles were observed. The overexpression of enolase 1, Eef2, and vinculin in 4T1.2 cells elevated the EdU-based proliferation and TRANSWELL® invasion of 4T1.2 cells, and the levels of Lep5, MMP9, Runx2, TGFβ, and Snail were elevated (FIG. 23A-C). However, the application of their recombinant proteins extracellularly to 4T1.2 cells reduced the EdU-based proliferation, TRANSWELL® invasion, and downregulated Lrp5, MMP9, Runx2, TGFβ, and Snail (FIG. 23D-F).

Regarding Hsp90ab1, the application of its recombinant proteins reduced TRANSWELL® invasion and downregulated Lrp5, MMP9, Runx2, TGFβ, and Snail (FIG. 23G&H). Also, in response to CM derived from 4T1.2 cells treated with siRNAs specific to enolase 1, Hsp90ab1, and Eef2, the scratch-based migration of 4T1.2 cells was stimulated and the levels of Lrp5, MMP9, Runx2, TGFβ, and Snail were elevated in 4T1.2 cells (FIG. 23A&B). Consistent with the elevation of Lrp5, MMP9, Runx2, TGFβ, and Snail, the levels of p53 and Trail were reduced (FIG. 23C). Taken together, the result showed that the four proteins such as enolase 1, Hsp90ab1, Eef2, and vinculin, which were enriched in iTS cell-derived CM, acted as tumor suppressors extracellularly and tumor promoters intracellularly.

Variations in tumor selectivity among the predicted tumor-suppressing proteins. Tumor-suppressing proteins may inhibit the progression of tumor cells but not inhibit the progression of non-tumor cells. Using the MTT-based viability, tumor selectivity was defined as a ratio of (reduction in MTT-based viability of tumor cells) to (reduction in MTT-based viability of non-tumor cells). The tumor selectivity may be above one to remove tumor cells and a larger value may signify a reduction in the inhibition of the growth of non-tumor cells. The tumor selectivity was determined using three tumor cells (4T1.2 mouse mammary tumor cells, EO771 mammary tumor cells, and MDA-MB-231 breast cancer cells) and three non-tumor cells (adipose cells, MLO-A5 osteocytes, and murine MSCs). The inhibitory effects of both Oct4 CM and c-Myc CM were selective to tumor cells, although Oct4 CM presented a higher selectivity than c-Myc CM. Table 2 provides the tumor selectivity of 4T1.2 Oct4 CM, 4T1.2 c-Myc CM, and 10 tumor-suppressing protein candidates. In Table 2, the term “N.D.” means that the value in question was not defined, because the adipose-derived MSCs actually increased in MTT-based viability, for example.

TABLE 2

| Tumor Cells | | |
|---|---|---|
| 4T1.2 | EO | MDA |

4T1.2 Oct4 CM

| | | | | |
|---|---|---|---|---|
| Non-tumor | Adipose | N.D. | N.D. | N.D. |
| Cells | A5 | 3.44 | 3.18 | 2.58 |
| | MSC | 4.27 | 3.94 | 3.20 |

4T1.2 c-Myc CM

| | | | | |
|---|---|---|---|---|
| Non-tumor | Adipose | 7.01 | 5.82 | 5.70 |
| Cells | A5 | 2.20 | 1.80 | 1.76 |
| | MSC | 3.21 | 2.64 | 2.60 |

Eno1

| | | | | |
|---|---|---|---|---|
| Non-tumor | Adipose | N.D. | N.D. | N.D. |
| Cells | A5 | 1.48 | 3.29 | 3.11 |
| | MSC | 3.30 | 7.34 | 6.94 |

Hsp90ab1

| | | | | |
|---|---|---|---|---|
| Non-tumor | Adipose | N.D. | N.D. | N.D. |
| Cells | A5 | 1.68 | 1.55 | 1.02 |
| | MSC | 2.86 | 2.65 | 1.74 |

Eef2

| | | | | |
|---|---|---|---|---|
| Non-tumor | Adipose | 2.05 | 2.93 | 1.78 |
| Cells | A5 | 2.67 | 3.51 | 3.49 |
| | MSC | 1.18 | 1.42 | 1.18 |

Aldoa

| | | | | |
|---|---|---|---|---|
| Non-tumor | Adipose | 3.06 | 2.10 | 1.97 |
| Cells | A5 | 2.72 | 1.87 | 1.75 |
| | MSC | 1.39 | 0.96 | 0.90 |

Flna

| | | | | |
|---|---|---|---|---|
| Non-tumor | Adipose | 1.19 | 1.64 | 1.28 |
| Cells | A5 | 1.43 | 1.97 | 1.54 |
| | MSC | 1.05 | 1.45 | 1.13 |

Plec

| | | | | |
|---|---|---|---|---|
| Non-tumor | Adipose | 1.28 | 1.86 | 1.10 |
| Cells | A5 | 1.33 | 1.95 | 1.16 |
| | MSC | 1.06 | 1.54 | 0.91 |

VCL

| | | | | |
|---|---|---|---|---|
| Non-tumor | Adipose | 1.43 | 1.27 | 1.88 |
| Cells | A5 | 0.99 | 0.67 | 1.13 |
| | MSC | 1.57 | 0.89 | 1.43 |

Eef1a1

| | | | | |
|---|---|---|---|---|
| Non-tumor | Adipose | 0.74 | 0.58 | 0.52 |
| Cells | A5 | 1.23 | 0.96 | 0.86 |
| | MSC | 1.34 | 1.05 | 0.94 |

Pfn1

| | | | | |
|---|---|---|---|---|
| Non-tumor | Adipose | 0.92 | 0.77 | 0.58 |
| Cells | A5 | 1.14 | 0.95 | 0.72 |
| | MSC | 1.05 | 0.87 | 0.66 |

Myh9

| | | | | |
|---|---|---|---|---|
| Non-tumor | Adipose | 0.48 | 0.64 | 0.46 |
| Cells | A5 | 0.25 | 0.32 | 0.23 |
| | MSC | 0.70 | 0.93 | 0.66 |

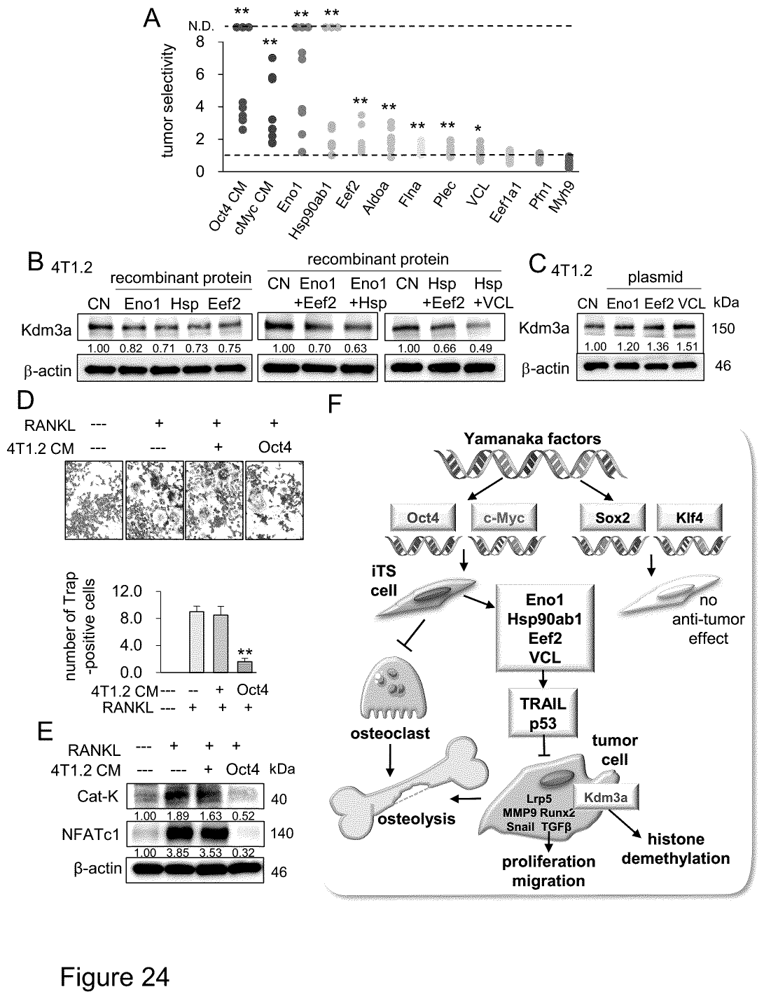

Interestingly, Oct4 CM, Eno1, and Hsp90ab1 elevated the MTT-based viability of non-tumor cells, such as adipose-derived MSCs, and their tumor selectivities were undefined. Among 10 tumor-suppressing protein candidates, 7 proteins such as enolase 1, Hsp90ab1, Eef2, Aldoa, Flna, Plec, and Vcl gave the tumor selectivity significantly higher than 1 (FIG. 24A).

Kdm3a as a target of enolase 1, Hsp90ab1, Eef2, and vinculin. It was shown that the expression of Kdm3a, lysine-specific demethylase for histones, in 4T1.2 tumor cells was downregulated by Oct4 CM and c-Myc CM. Consistently, the administration of enolase 1, Hsp90ab1, Eef2, and vinculin reduced the level of Kdm3a in 4T1.2 cells. Also, several cases showed that a combinatorial administration of these proteins enhanced the reduction of Kdm3a (FIG. 24B). In contrast, the overexpression of enolase 1, Eef2, and vinculin in 4T1.2 cells elevated the level of Kdm3a (FIG. 24C). The result suggests that histone methylation might be altered by the newly identified extracellular tumor suppressors, which were enriched in CM by the overexpression of Oct4 and c-Myc and not by the overexpression of Sox2 and Klf4.

Suppression of osteoclast development. In tumor-invaded bone, bone-resorbing osteoclasts play a role in bone destruction, and blocking their development may be important. The effect of Oct4 CM on the maturation of RAW264.7 pre-osteoclasts was examined. The result revealed that Oct4 CM inhibited the differentiation of RANKL-stimulated pre-osteoclasts and downregulated two key regulators, cathepsin K as a bone-resorbing protease and NFATc1 as a master transcription factor of osteoclastogenesis (FIG. 24D&E). The result indicates that iTS cell-derived CM may repress the vicious bone-degradation cycle in the bone microenvironment by reducing not only tumor progression but also osteoclast development.

Other variations or embodiments will be apparent to a person of ordinary skill in the art from the above-description. Thus, the foregoing embodiments are not to be construed as limiting the scope of the claimed subject matter. All references disclosed are expressly incorporated by reference in in their entirety.

### REFERENCES

Each of the following documents is hereby expressly incorporated by reference herein in its entirety.
Akieda Y, Ogamino S, Furuie H, Ishitani S, Akiyoshi R, Nogami J, et al. Cell competition corrects noisy Wnt morphogen gradients to achieve robust patterning in the zebrafish embryo. Nat Commun. 2019; 10(1):4710.
An T, Qin S, Xu Y, Tang Y, Huang Y, Situ B, et al. Exosomes serve as tumour markers for personalized diagnostics owing to their important role in cancer metastasis. J Extracell Vesicles. 2015; 4:27522.
Au K M, Wang A Z, and Park S I. Pretargeted delivery of PI3K/mTOR small-molecule inhibitor-loaded nanoparticles for treatment of non-Hodgkin's lymphoma. Sci Adv. 2020; 6(14): eaaz9798.
Balogh G A, Mailo D A, Corte M M, Roncoroni P, Nardi H, Vincent E, et al. Mutant p53 protein in serum could be used as a molecular marker in human breast cancer. Int J Oncol. 2006; 28(4):995-1002.
Barbara A. Malynn and Averil Ma. Ubiquitin makes its mark on immune regulation. Immunity 2010; 33(6): 843-852.
Baron R, and Kneissel M. WNT signaling in bone homeostasis and disease: from human mutations to treatments. Nat Med. 2013; 19(2):179-92.
Cappellen D, Schlange T, Bauer M, Maurer F, Hynes N E. Novel c-MYC target genes mediate differential effects on cell proliferation and migration. EMBO Rep. 2007 January; 8(1):70-6.
Chi V Dang. MYC on the Path to Cancer. Cell. 2012 Mar. 30; 149(1):22-35.
Chiou S H, Wang M L, Chou Y T, Chen C J, et al. Coexpression of Oct4 and Nanog enhances malignancy in lung adenocarcinoma by inducing cancer stem cell-like properties and epithelial-mesenchymal transdifferentiation. Cancer Res. 2010 Dec. 15; 70(24):10433-44.
Choi B K, Dayaram T, Parikh N, Wilkins A D, Nagarajan M, Novikov I B, et al. Literature-based automated discovery of tumor suppressor p53 phosphorylation and inhibition by NEK2. Proc Natl Acad Sci USA. 2018; 115(42): 10666-71.
Ciacma K, Wieckiewicz J, Kedracka-Krok S, Kurtyka M, Stec M, Siedlar M, et al. Secretion of tumoricidal human tumor necrosis factor-related apoptosis-inducing ligand (TRAIL) by recombinant Lactococcus lactis: optimization of in vitro synthesis conditions. Microb Cell Fact. 2018; 17(1):177.
Clancy L, Mruk K, Archer K, Woelfel M, Mongkolsapaya J, Screaton G, et al. Preligand assembly domain-mediated ligand-independent association between TRAIL receptor 4 (TR4) and TR2 regulates TRAIL-induced apoptosis. Proc Natl Acad Sci USA. 2005; 102(50): 18099-104.
Connelly, K. E., V. Hedrick, T. J. Paschoal Sobreira, E. C. Dykhuizen and U. K. Aryal. Analysis of Human Nuclear Protein Complexes by Quantitative Mass Spectrometry Profiling. Proteomics 2018; 18(11): e1700427.
Cox, J., and M. Mann (2008). “MaxQuant enables high peptide identification rates, individualized p.p.b.-range mass accuracies and proteome-wide protein quantification.” Nat Biotechnol 26(12): 1367-1372.
Dandawate P, Chandrayee Ghosh, Kanagaraj Palaniyandi, Santanu Paul, et al. The Histone Demethylase KDM3A, Increased in Human Pancreatic Tumors, Regulates Expression of DCLK1 and Promotes Tumorigenesis in Mice Gastroenterology. 2019 December; 157(6):1646-1659.e11. doi: 10.1053/j.gastro.2019.08.018. Epub 2019 Aug. 20.
Dingding Shi and Steven R Grossman. Ubiquitin becomes ubiquitous in cancer—Emerging roles of ubiquitin ligases and deubiquitinases in tumorigenesis and as therapeutic targets. Cancer Biol Ther. 2010; 10(8): 737-747.
Erbayraktar Z, Alural B, Erbayraktar R S, and Erkan E P. Cell division cycle 7-kinase inhibitor PHA-767491 hydrochloride suppresses glioblastoma growth and invasiveness. Cancer Cell Int. 2016; 16:88.
Ewens A, Mihich E, Ehrke M J. Distant metastasis from subcutaneously grown EO771 medullary breast adenocarcinoma. Anticancer Res. 2005; 25(6B):3905-15.
Fan Y, Jalali A, Chen A, Zhao X, Liu S, Teli M, et al. Skeletal loading regulates breast cancer-associated osteolysis in a loading intensity-dependent fashion. Bone Res. 2020; 8:9.
Faubert B, Solmonson A, and DeBerardinis R J. Metabolic reprogramming and cancer progression. Science. 2020; 368(6487).
Fernando Faunes, Penelope Hayward, Silvia Munoz Descalzo, Suj ash S Chatterjee, et al. A Membrane-Associated β-catenin/Oct4 Complex Correlates With Ground-State Pluripotency in Mouse Embryonic Stem Cells. Development. 2013 March; 140(6):1171-83.
Frampton A E, Prado M M, Lopez-Jimenez E, Fajardo-Puerta A B, Jawad Z A R,
Lawton P, et al. Glypican-1 is enriched in circulating-exosomes in pancreatic cancer and correlates with tumor burden. Oncotarget. 2018; 9(27):19006-13.
Fulda S. Tumor-necrosis-factor-related apoptosis-inducing ligand (TRAIL). Adv Exp Med Biol. 2014; 818:167-80.
Gasser S M. Selfish DNA and Epigenetic Repression Revisited. Genetics. 2016; 204(3):837-9.
Ghosh N, Hossain U, Mandal A, and Sil P C. The Wnt signaling pathway: a potential therapeutic target against cancer. Ann N Y Acad Sci. 2019; 1443(1):54-74.
Goldman M, Craft B, Hastie M, Repeela K, McDade F, Kamath A, et al. The UCSC Xena platform for public and private cancer genomics data visualization and interpretation. bioRxiv. 2019.
Goloudina A R, Tanoue K, Hammann A, Fourmaux E, Le Guezennec X, Bulavin D V, et al. Wip1 promotes RUNX2-dependent apoptosis in p53-negative tumors and protects normal tissues during treatment with anticancer agents. Proc Natl Acad Sci USA. 2012; 109(2): E68-75.
Groza M, Zimta A A, Irimie A, Achimas-Cadariu P, Cenariu D, Stanta G, et al. Recent advancements in the study of breast cancer exosomes as mediators of intratumoral communication. J Cell Physiol. 2020; 235(2): 691-705.
Hamamura K., et al. Knee loading reduces MMP13 activity in the mouse cartilage. BMC Musculoskelet Disord 2013; 14:312.
Howe, E. N., M. D. Burnette, M. E. Justice, P. M. Schnepp, V. Hedrick, J. W. Clancy, I. H. Guldner, A. T. Lamere, J. Li, U. K. Aryal, C. D'Souza-Schorey, J. J. Zartman and S. Zhang. Rab11b-mediated integrin recycling promotes brain metastatic adaptation and outgrowth. Nat Commun 2020; 11(1): 3017.
Hsu J L, Pan S L, Ho Y F, Hwang T L, Kung F L, Guh J H. Costunolide induces apoptosis through nuclear calcium2+ overload and DNA damage response in human prostate cancer. J Urol 2011; 185: 1967-1974.
Huang H. Matrix Metalloproteinase-9 (MMP-9) as a Cancer Biomarker and MMP-9 Biosensors: Recent Advances. Sensors (Basel). 2018; 18(10).
Hyun Jung Kim, Venkataraman Magesh, et al. Ubiquitin C-terminal hydrolase-L1 increases cancer cell invasion by modulating hydrogen peroxide generated via NADPH oxidase 4. Oncotarget 2015 Jun. 30; 6(18):16287-303.
Jiang C, Xu R, Li X X, Zhou Y F, Xu X Y, Yang Y, et al. Sorafenib and Carfilzomib Synergistically Inhibit the Proliferation, Survival, and Metastasis of Hepatocellular Carcinoma. Molecular cancer therapeutics. 2018; 17(12):2610-21.
Johnstone C N, Pattison A D, Harrison P F, Powell D R, Lock P, Ernst M et al. FGF13 promotes metastasis of triple-negative breast cancer. Int J Cancer 2020.
Kalluri R. The biology and function of exosomes in cancer. The Journal of clinical investigation. 2016; 126(4): 1208-15.
Kroger C, Afeyan A, Mraz J, Eaton E N, Reinhardt F, Khodor Y L, et al. Acquisition of a hybrid ENI state is essential for tumorigenicity of basal breast cancer cells. Proc Natl Acad Sci USA. 2019; 116(15):7353-62.
Li Cheng Jin Zang Han-Jue Dai Feng Li Feng Guo. Ubiquitin ligase CHIP functions as an oncogene and activates the AKT signaling pathway in prostate cancer. Int J Oncol. 2018; 53(1):203-214.
Li F, Chen A, Reeser A, Wang Y, Fan Y, Liu S et al. Vinculin Force Sensor Detects Tumor-Osteocyte Interactions. Sci Rep 2019; 9: 5615.
Liu J, Wu X, Mitchell B, Kintner C, Ding S, and Schultz P G. A small-molecule agonist of the Wnt signaling pathway. Angew Chem Int Ed Engl. 2005; 44(13):1987-90.
Liu S, Fan Y, Chen A, Jalali A, Minami K, Ogawa K, et al. Osteocyte-driven downregulation of snail restrains effects of Drd2 inhibitors on mammary tumor cells. Cancer Res. 2018; 78(14): 3865-3876. doi:10.1158/0008-5472.CAN-18-0056.
Liu S, Liu Y, Minami K, Chen A, Wan Q, Yin Y, et al. Inhibiting checkpoint kinase 1 protects bone from bone resorption by mammary tumor in a mouse model. Oncotarget. 2018; 9(10):9364-9378. doi:10.18632/oncotarget.24286.
Maeda K, Kobayashi Y, Koide M, Uehara S, Okamoto M, Ishihara A, et al. The Regulation of Bone Metabolism and Disorders by Wnt Signaling. Int J Mot Sci. 2019; 20(22).
Marloes Hoeksema, Martin van Eijk, Henk P Haagsman, and Kevan L Hartshorn. Histones as mediators of host defense, inflammation and thrombosis. Future Microbiol. 2016; 11(3): 441-453.
Merino M M, Levayer R, and Moreno E. Survival of the Fittest: Essential Roles of Cell Competition in Development, Aging, and Cancer. Trends Cell Biol. 2016; 26(10):776-88.
Miller D M, Thomas S D, Islam A, Muench D, Sedoris K. c-Myc and cancer metabolism. Clin Cancer Res. 2012 Oct. 15; 18(20):5546-53.
Minami K, Liu S, Liu Y, Chen A, Wan Q, Na S, et al. Inhibitory Effects of Dopamine Receptor D1 Agonist on Mammary Tumor and Bone Metastasis. Sci Rep. 2017; 7: 45686. doi:10.1038/srep45686.
Naval J, de Miguel D, Gallego-Lleyda A, Anel A, and Martinez-Lostao L. Importance of TRAIL Molecular Anatomy in Receptor Oligomerization and Signaling. Implications for Cancer Therapy. Cancers (Basel). 2019; 11(4).
Ni D, Liu J, Hu Y, Liu Y, Gu Y, Zhou Q, et al. A1CF-Axin2 signal axis regulates apoptosis and migration in Wilms tumor-derived cells through Wnt/beta-catenin pathway. In Vitro Cell Dev Biol Anim. 2019; 55(4):252-9.
Novak D, Wiser L, Elton J J, Umansky V, Altevogt P, Utikal J. SOX2 in development and cancer biology. Semin Cancer Biol. 2019 Aug. 11.
Oh Y S, Kim H Y, Song I C, Yun H J, Jo D Y, Kim S, et al. Hypoxia induces CXCR4 expression and biological activity in gastric cancer cells through activation of hypoxia-inducible factor-1 alpha. Oncol Rep. 2012; 28(6):2239-46.
Olivero C E, Martinez-Terroba E, Zimmer J, Liao C, Tesfaye E, Hooshdaran N, et al. p53 Activates the Long Noncoding RNA Pvt1b to Inhibit Myc and Suppress Tumorigenesis. Mol Cell 2020; 77(4):761-74 e8.
Opoku-Temeng, C., K. I. Onyedibe, U. K. Aryal, and H. O. Sintim (2019). “Proteomic analysis of bacterial response to a 4-hydroxybenzylidene indolinone compound, which re-sensitizes bacteria to traditional antibiotics.” J Proteomics 202: 103368.
Parker T M, Henriques V, Beltran A, Nakshatri H, and Gogna R. Cell competition and tumor heterogeneity. Seminars in cancer biology. 2019.
Pham P V, Le H T, Vu B T, Pham V Q, Le P M, Phan N L, et al. Targeting breast cancer stem cells by dendritic cell vaccination in humanized mice with breast tumor: preliminary results. Onco Targets Ther. 2016; 9:4441-4451. doi:10.2147/OTT. S105239
Polakis P. Wnt signaling in cancer. Cold Spring Harb Perspect Biol. 2012; 4(5).
Price P J. Best practices for media selection for mammalian cells. In Vitro Cell Dev Biol Anim. 2017; 53(8):673-81.
Qian Z, and Adhya S. DNA repeat sequences: diversity and versatility of functions. Curr Genet. 2017; 63(3):411-6.
Quail D F, and Joyce J A. Microenvironmental regulation of tumor progression and metastasis. Nat Med. 2013; 19(11): 1423-37.
Ramalingam P, Poulos M G, Lazzari E, Gutkin M C, Lopez D, Kloss C C, et al. Chronic activation of endothelial MAPK disrupts hematopoiesis via NFKB dependent inflammatory stress reversible by SCGF. Nat Commun. 2020; 11 (1):666.
Saha S K, Jeong Y, Cho S, Cho S G. Systematic expression alteration analysis of master reprogramming factor OCT4 and its three pseudogenes in human cancer and their prognostic outcomes. Sci Rep. 2018 Oct. 4; 8(1):14806
Scudellari M. How iPS cells changed the world. Nature. 2016 Jun. 16; 534(7607):310-2.
Seo H K, Lee S J, Kwon W A, and Jeong K C. Docetaxel-resistant prostate cancer cells become sensitive to gemcitabine due to the upregulation of ABCB1. Prostate 2020; 80(6):453-62.
Sun J, Liu Y, Chen Y, Zhao W, Zhai Q, Rathod S et al. Doxorubicin delivered by a redox-responsive dasatinib-containing polymeric prodrug carrier for combination therapy. J Control Release 2017; 258: 43-55.
Takahashi K, Yamanaka S. Induction of pluripotent stem cells from mouse embryonic and adult fibroblast cultures by defined factors. Cell. 2006 Aug. 25; 126(4):663-76.
Takigawa S, Frondorf B, Liu S, Liu Y, Li B, Sudo A, et al. Salubrinal improves mechanical properties of the femur in osteogenesis imperfecta mice. J Pharmacol Sci. 2016; 132(2): 154-61.
Thorpe L M, Yuzugullu H, and Zhao J J. PI3K in cancer: divergent roles of isoforms, modes of activation and therapeutic targeting. Nat Rev Cancer. 2015; 15(1):7-24.
Tilborghs S, Corthouts J, Verhoeven Y, Arias D, Rolfo C, Trinh X B, et al. The role of Nuclear Factor-kappa B signaling in human cervical cancer. Crit Rev Oncol Hematol. 2017; 120:141-50.
VictoriaRadon VictoriaRadón, Maire Czesla, Julia Reichelt, Julia Fehlert, Anna Hammel, Alva Rosendahl. Ubiquitin C-Terminal Hydrolase L1 is required for regulated protein degradation through the ubiquitin proteasome system in kidney. Implications for Cancer Therapy. Kidney Int. 2018 January; 93(1): 110-127.
Wang Y, Shi J, Chai K, Ying X, and Zhou B P. The Role of Snail in EMT and Tumorigenesis. Curr Cancer Drug Targets 2013; 13 (9): 963-72.
Wu G, Scholer H R. Role of Oct4 in the early embryo development. Cell Regen (Lond). 2014 Apr. 29; 3(1):7.
Xin Yong, Bo Tang, Yu-Feng Xiao, Rui Xie, Yong Qin, et al. Helicobacter Pylori Upregulates Nanog and Oct4 via Wnt/β-catenin Signaling Pathway to Promote Cancer Stem Cell-Like Properties in Human Gastric Cancer. Cancer Lett. 2016 May 1; 374(2):292-303.
Xu W, Wan Q, Na S, Yokota H, Yan J. L, and Hamamura K. Suppressed invasive and migratory behaviors of SW1353 chondrosarcoma cells through the regulation of Src, Rac1 GTPase, and MMP13. Cell Signal. 2015; 27(12):2332-42. doi:10.1016/j.cellsig.2015.08.014.
Yu F, Li J, Chen H, Fu J, Ray S, Huang S, Zheng H, Ai W. Kruppel-like factor 4 (KLF4) is required for maintenance of breast cancer stem cells and for cell migration and invasion. Oncogene. 2011 May 5; 30(18):2161-72.
Yu X, Harris S L, and Levine A J. The regulation of exosome secretion: a novel function of the p53 protein. Cancer research 2006; 66(9):4795-801.

1. A pharmaceutical composition comprising
a cell-free conditioned medium (CM) or extract or concentrate thereof obtained from a mammalian cell culture medium comprising a cultured substantially homogenous cancerous mammalian cell population;
wherein at least a portion of the cancerous mammalian cell population is contacted by at least one small molecule cell growth signaling pathway activator before being cultured in the cell culture medium.

2. (canceled)

3. The pharmaceutical composition of claim 1, wherein the at least a portion of the cancerous mammalian cell population is contacted by at least two small molecule cell growth signaling pathway activators before being cultured in the cell culture medium.

4. The pharmaceutical composition of claim 1, further comprising a pharmaceutically acceptable carrier.

5. The pharmaceutical composition of claim 1, wherein the conditioned medium further comprises a cancerous mammalian cell-secreted protein selected from the group consisting of heat shock protein 90 alpha family class B member 1 (Hsp90ab1), enolase 1 (Eno1), eukaryotic translation elongation factor 2 (Eef2), ubiquitin C (Ubc), and vinculin (VCL).

6. The pharmaceutical composition of claim 1, wherein the composition is enriched with a cancerous mammalian cell-secreted protein selected from the group consisting of heat shock protein 90 alpha family class B member 1 (Hsp90ab1), enolase 1 (Eno1), eukaryotic translation elongation factor 2 (Eef2), ubiquitin C (Ubc), and vinculin (VCL).

7. The pharmaceutical composition of claim 1, further comprising a chemotherapeutic agent.

8. The pharmaceutical composition of claim 1, wherein the small molecule cell growth signaling pathway activator is a small molecule Wnt signaling pathway activator.

9. The pharmaceutical composition of claim 8, wherein the small molecule Wnt signaling pathway activator is BML-284, or a pharmaceutically acceptable salt thereof.

10. The pharmaceutical composition of claim 1, wherein the cancerous mammalian cells are cancerous mammalian bone cells.

11. (canceled)

12. (canceled)

13. A kit comprising:
a pharmaceutical composition comprising
a cell-free conditioned medium (CM) or extract or concentrate thereof obtained from a mammalian cell culture medium comprising a cultured substantially homogenous cancerous mammalian cell population;
wherein at least a portion of the cancerous mammalian cell population is contacted by at least one small molecule cell growth signaling pathway activator before being cultured in the cell culture medium;
a container;
a label; and
instructions that provide methods for administering the composition.

14. (canceled)

15. A method to treat a cancer in a subject in need thereof, the method comprising:
administering to the subject in need thereof a therapeutically effective amount of a cell-free conditioned medium (CM) or extract or concentrate thereof obtained from a mammalian cell culture medium comprising a cultured substantially homogenous cancerous mammalian cell population;
wherein a portion of the cancerous mammalian cell population is contacted by a small molecule cell growth signaling pathway activator before being cultured in the cell culture medium.

16. (canceled)

17. The method of claim 15, wherein the treated cancer is a metastatic cancer and the cultured substantially homogenous cancerous mammalian cell population is derived from a same organ or tissue as the organ or site to be treated.

18. (canceled)

19. (canceled)

20. The method of claim 15, wherein the treated cancer is a metastatic bone cancer.

21. The method of claim 15, wherein the treated cancer is a primary cancer.

22. (canceled)

23. (canceled)

24. (canceled)

25. The method of claim 15, wherein the small molecule cell growth signaling pathway activator is selected from the group consisting of a small molecule Wnt signaling pathway activator, a small molecule PI3K signaling pathway activator, a small molecule FGF signaling pathway activator and a small molecule Notch signaling pathway activator.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 15, wherein the method results in at least one activity selected from the group consisting of upregulating a tumor-suppressing gene in a cancer cell, downregulating a tumor-promoting gene in a cancer cell, inhibiting cancer cell invasion, inhibiting cancer cell growth, and inhibiting cancer cell recurrence.

41. The method of claim 15, wherein the target cancer is a therapy-resistant cancer.

42. A process to produce a conditioned medium (CM), the process comprising:
contacting cancerous mammalian cells with a small molecule cell growth signaling pathway activator to generate pre-treated cancerous mammalian cells;
culturing the pre-treated cancerous mammalian cells in a mammalian cell culture medium for a period of time sufficient to condition the medium;
removing the pre-treated cancerous mammalian cells from the culture medium; and,
collecting the conditioned medium.

43. The process of claim 42, further comprising centrifuging the conditioned medium to remove exosomes.

44. The process of claim 42, wherein the mammalian cell culture medium is serum-free.

45. The process of claim 42, wherein the pre-treated cancerous mammalian cells are cultured for a time period from about 1 hour to about 24 hours.

46. The process of claim 42, further comprising filtering the collected conditioned medium.

47. (canceled)

48. The process of claim 42, further comprising concentrating the collected conditioned medium.

49. (canceled)

50. (canceled)

51. (canceled)
